(19) United States
(12) Patent Application Publication
(10) Pub. No.: US 2025/0212873 A1
(43) Pub. Date: Jul. 3, 2025

(54) VOLATILE HDAC INHIBITORS FOR THERAPEUTIC AND PLANT APPLICATIONS

(71) Applicant: The Regents of the University of California, Oakland, CA (US)

(72) Inventors: Anandasankar Ray, Riverside, CA (US); Rogelio Junior Nunez-Flores, Perris, CA (US)

(73) Assignee: The Regents of the University of California, Oakland, CA (US)

(21) Appl. No.: 18/852,384

(22) PCT Filed: Mar. 30, 2023
(86) PCT No.: PCT/US2023/016808

§ 371 Date: Sep. 27, 2024

Related U.S. Application Data
(60) Provisional application No. 63/325,468, filed on Mar. 30, 2022.

(51) Int. Cl. A01N 35/02; A01P 13/00; A01P 21/00; A61K 31/121; A61P 35/00; G01N 33/00

(52) U.S. Cl. CPC A01N 35/02; A01P 13/00; A01P 21/00; A61K 31/121; A61P 35/00; G01N 33/0098

(57) ABSTRACT

The invention provides volatile HD AC inhibiting compounds for agricultural and medicinal applications.

### CROSS-REFERENCE TO RELATED APPLICATION

This application claims priority to U.S. Provisional Application No. 63/325,468 filed on Mar. 30, 2022. The entire content of the application referenced above is hereby incorporated by reference herein.

### BACKGROUND

Histone deacetylases (HDAC) are a class of enzymes that remove acyl groups from ϵ-N-acetyl lysine amino acids on a histone. This is important because DNA expression is regulated by acetylation and de-acetylation through post translational modification of histones and other cellular proteins. The HDAC family members are highly conserved, spanning both animal and plant kingdoms. Eukaryotic DNA is wound around histone protein octamers typically comprised of the core histone proteins, H2A, H2B, H3 and H4.

Eleven (11) subtypes of HDACs have been identified in mammals, which fall into class I (HDAC1, HDAC2, HDAC3, HDAC8), class IIa (HDAC4, HDAC5, HDAC7, HDAC9) and class IIb (HDAC6, HDAC10) subgroups, that are distinct from class IV which only includes HDAC11.

The structure and function of the DNA-histone chromatin complex is altered by dynamic post-translational modifications on the basic histone tails (hPTMs). Lysine acetylation (ac) and methylation, two among many such hPTMs, play key roles in regulating gene expression, and involve a complex “language” of spatial patterns that regulate chromatin state, such as open or closed. Such epigenetic changes influence gene expression without altering the genome, enabling transcriptional plasticity while maintenance of cell identity. Epigenetic regulation can also have a “memory” in that chromatin state can be retained for exceedingly long times in post-mitotic cells, such as neurons.

Due to their impact on gene regulation, HDACs are considered promising targets for drug development for many diseases. Several classes of HDAC inhibitors, when administered orally, have been found to attenuate the progression of a repertoire of neurodegenerative disorders and cancers. While high potency HDAC inhibitors are an important class of drug-like molecules in FDA trials for brain cancers they pose challenges related to side effects and crossing the blood brain barrier. In mammals, the olfactory epithelium-lined nasal pathway likely provides a trans-epithelial transport route for small molecules to bypass the blood-brain barrier and enter the central nervous system, thus presenting a natural delivery system to neurons in the brain.

HDACs play a role in various developmental, and stress related factors and their inhibitors can have several agricultural applications. HDAC inhibitors also have several therapeutic applications. For example, HDAC inhibitors are useful for: 1) treating or delaying the development of a cancer, 2) treating or delaying the development of Alzheimer's disease, 3) improving immune response, 4) modulating gene expression in brain neurons, past the blood brain barrier, and 5) treating or delaying the development of chronic obstructive pulmonary disorder (COPD).

In plants several important signaling pathways use VOCs, such as methyl salicylate, jasmonic acid, and ethylene, which cause a variety of downstream effects including changes in gene expression (L. L. Walling, The Myriad Plant Responses to Herbivores. J Plant Growth Regul 19, 195-216 (2000)). Plants are known to have systemic responses to many other volatile chemicals too, which alter growth or immune responses (S. Lee, et al., Fungal Ecology 37, 1-9 (2019)). Both monocotyledonous and dicotyledonous species with herbivorous insect damage release aboveground VOCs, which alter neighboring plants (M. Heil, R. Karban, Trends Ecol Evol 25, 137-144 (2010)). Moreover, both intra and interspecies forms of communication via VOCs exist. Plant roots also produce VOCs, as do the plant rhizobiome, and microbes living in the soil (R. J. Morcillo et al., EMBO J 39, e102602 (2020); U. Effmert, et al., J Chem Ecol 38, 665-703 (2012); M. A. Farag, et al., Phytochemistry 67, 2262-2268 (2006); and R. Ortiz-Castro, et al., Plant Signal Behav 4, 701-712 (2009)).

Volatile microbial metabolites have been present throughout the evolution of plants (M. McFall-Ngai et al., Proceedings of the National Academy of Sciences 110, 3229-3236 (2013)) and are potential signals for multi-domain communication because they can travel wide ranges. Plants are known to sense microbial metabolites from root infections and control timing of their immune response (G. Effantin, et al., Molecular microbiology 82, 988-997 (2011)) and microbial volatiles also elicit olfactory behaviors in insects and nematodes (24, 25), and other bacteria (B. Audrain, et al., FEMS Microbiology Reviews, fuu013 (2015); and K. -S. Kim, et al., Nature Communications 4, 1809 (2013)). Eukaryotes in particular have evolved very diverse trans-membrane odor receptor families, but several, like plants, even lack neurons. HDAC proteins, however, are an ancient family of genes that predate even histones themselves (I. V. Gregoretti, et al., J Mol Biol 338, 17-31 (2004); J. Postberg, et al., BMC Evol Biol 10, 259 (2010); and D. D. Leipe, et al., Nucleic Acids Res 25, 3693-3697 (1997)).

Currently, there is a need for novel HDAC inhibiting compounds that can be used in agricultural and/or therapeutic applications. In particular, there is a need for simple, readily available HDAC inhibiting compounds and for HDAC inhibiting compounds that have low molecular weights or are volatile and can enter the target tissues like leaves, roots in plants and across the blood-brain barrier in animals.

### SUMMARY

The Applicant has identified a series of simple, readily-available HDAC inhibiting compounds that are useful for both agricultural and therapeutic applications. Many of the HDAC inhibiting compounds are volatile, which differentiates the compounds from other known HDAC inhibitors. When inhaled from low levels in the environment, volatile HDAC inhibitors were found to alter gene expression in the mouse brain, presumably due to alteration in chromatin.

In one aspect the invention provides a method of altering or delaying development of plant tissue, or response to abiotic stress, or infection in a plant in need thereof, comprising contacting the plant with an effective amount of 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, methylglyoxal, benzyl nicotinate, a flavone, phenethyl resorcinol, acacetin, eriodictyol, phloretin, 2-phenylethyl 2-aminobenzoate, aromadedrin, benzoyl peroxide, heliotropyl phenyl acetate, chalcone, benzyl para-anisate, cinnamyl anthranilate, (Z)-2-penten-1-yl benzoate, 2-naphthyl phenyl ketone, bergamottin, isoimperatorin, para-cresyl benzoate, guaiacyl benzoate, caffeic acid ethyl ester, 4-phenyl propyl pyridine, 2-phenylethyl benzoate, benzyl benzoate, 2-methyl butyl salicylate, phenethyl sorbate, para-anisaldehyde, methyl anthranilate schiff's base, bibenzyl ether, ortho-anisyl benzoate, cinnamyl benzoate, 1-(2-hydroxy-4-methoxyphenyl)-3-(pyridin-2-yl) propan-1-one, ethyl 4-methylphenoxyacetate, benzyl trans-2-methyl-2-butenoate, alpha,alpha-dimethyl anisyl acetone, styryl acetate, amyl cinnamate, ortho-cresyl benzoate, methyl phenyl methylene aminobenzoate, phenethyl pyridine, (Z)-N-coumaroyl-5-hydroxyanthranilic acid, homoeriodictyol, benzyl 2,3-dimethyl-2-butenoate, meta-cresyl benzoate, (Z)-anisylidene acetone, guaiacyl phenyl acetate, 3-phenyl propyl salicylate, geranyl cantabiline, cortex pyridine, ethyl para-cresyl carbonate, ethyl phenoxyacetate, hesperetin, 3′,7-dihydroxy-4′-methoxyflavan, allyl (Z)-cinnamate, bisdemethoxycurcumin, amyl salicylate, phenyl acetaldehyde, methyl anthranilate schiff's base, p-tolyl phenylacetate, 4-formyl phenyl butyrate, 4-methoxybenzyl phenylacetate, para-cresyl 3-oxo-3-phenyl propionate, (E)-2-hexen-1-yl salicylate, furfuryl phenyl acetate, butyl 2-naphthyl ether, (E)-cinnamyl acetate, cinnamyl acetate, ethyl (E)-cinnamate, ethyl cinnamate, 2-para-cresyl oxyethyl acetate, neryl anthranilate, cinnamaldehyde, methyl anthranilate schiff's base, cinnamyl tiglate, (E)-cinnamyl propionate, 1-(4-Methoxyphenyl)-1-penten-3-one, imperatorin, para-cresyl valerate, meta-cresyl phenyl acetate, 3-methyl-3-buten-1-yl benzoate, para-anisyl acetoacetate, isoamyl p-methoxycinnamate, isoeugenyl benzoate, (Z)-3-hexen-1-yl cinnamate, eugenyl benzoate, isoamyl salicylate, phenylmethyl benzeneacetate, curcumin, butyl cinnamate, cinnamyl acetoacetate, para-cresyl phenyl ether, citral, methyl anthranilate schiff's base, cinnamyl valerate, xanthohumol, allyl phenoxyacetate, 2-phenylethyl 3-phenyl-2-propenoate, geranyl benzoate, (E)-3-butylidene phthalide, geranyl cinnamate, neryl cinnamate, cinnamyl cinnamate, amyl anthranilate, (E)-2-hexen-1-yl formate, (E)-3-hexen-1-yl formate, (E)-9-tetradecen-1-yl formate, (E)-cinnamyl formate, (E,Z)-2,6-nonadien-1-yl formate, (Z)-3-hexen-1-yl formate, (Z)-9-tetradecen-1-yl formate, 1,3,4,5,6,7-hexahydro-1,1,5,5-tetramethyl-2H-2,4a-methanonaphthalen-7-yl formate, 1-hexyl allyl formate, 2,6-dimethylhept-5-enyl formate, 2-decalinyl formate, 2-melozol formate, 2-methoxyethyl formate, 2-methyl butyl formate, 2-naphthyl formate, 2-octyl formate, 2-pentyl formate, 2-phenoxyethyl formate, 3-cyclohexyl 3-hexenyl formate, 3-mercapto-3-methyl butyl formate, 3-octyl formate, 3-phenyl propyl formate, 4-tert-amyl cyclohexyl formate, 4-tert-butyl cyclohexyl formate, allyl formate, alpha-amyl cinnamyl formate, alpha-campholenyl formate, aluminum triformate, amber formate, ammonium formate, amyl formate, benzyl formate, bornyl formate, butyl chloroformate, butyl formate, calcium formate, carvyl formate, caryophyllene formate, cedryl formate, chitosan formate, cholesteryl formate, cinnamyl formate, citronellyl formate, cuminyl formate, cyclodecyl formate, cyclododecyl formate, cyclohexyl formate, cyclooctyl formate, cymbopogon martini herb extract formate, decahydro-1,1,7-trimethyl-3a,7-methano-3ah-cyclopentacyclooct-3-yl formate, decyl formate, dextro-bornyl formate, dihydrocarvyl formate, dihydrocitronellyl formate, dihydromyrcenyl formate, dimethyl benzyl carbinyl formate, dodecyl formate, ethyl 4-methyl-5-thiazolyl formate, ethyl benzoyl formate, ethyl chloroformate, ethyl formate, ethyl orthotrithioformate, ethyl vanillyl formate, eugenyl formate, fenchyl formate, furfuryl formate, geranyl formate, heliotropyl formate, heptyl formate, herbal cyclohexane formate, hexahydromethanoindenyl formate, hexahydrotetramethyl methanonaphthalene-8-methyl formate, hexyl formate, isoamyl formate, isobornyl formate, isobutyl formate, isodecyl formate, isoeugenyl formate, isononyl formate, isopropyl formate, isopulegyl formate, laevo-menthyl formate, linalyl formate, longifolene formate, marine formate, melozol formate, menthadienyl formate, menthyl formate, methyl formate, myristyl formate, myrtenyl formate, neryl formate, nonan-3-yl formate, nonisyl formate, nonyl formate, nopyl formate, octyl formate, oleyl formate, (±)-3-methyl-gamma-decalactone, (±)-chrysanthemolactone, (±)-dihydromint lactone, (±)-isoalantolactone, (±)-pantolactone, (+)-trans-whiskey lactone, (+/−)-mint lactone, (−)-cis-whiskey lactone, (−)-isomint lactone, (−)-mint lactone, (1R)-chrysanthemolactone, (1S)-chrysanthemolactone, (E)-dairy lactone, (R)-(−)-pantolactone, (R)-delta-decalactone, (R)-delta-dodecalactone, (R)-delta-hexalactone, (R)-delta-nonalactone, (R)-delta-octalactone, (R)-delta-undecalactone, (R)-epsilon-decalactone, (R)-gamma-decalactone, (R)-gamma-dodecalactone, (R)-gamma-heptalactone, (R)-gamma-hexalactone, (R)-gamma-nonalactone, (R)-gamma-octalactone, (R)-gamma-undecalactone, (R)-gamma-valerolactone, (R)-massoia lactone, (S)-delta-decalactone, (S)-delta-dodecalactone, (S)-delta-hexalactone, (S)-delta-nonalactone, (S)-delta-octalactone, (S)-delta-undecalactone, (S)-epsilon-decalactone, (S)-gamma-decalactone, (S)-gamma-dodecalactone, (S)-gamma-heptalactone, (S)-gamma-hexalactone, (S)-gamma-nonalactone, (S)-gamma-octalactone, (S)-gamma-undecalactone, (S)-gamma-valerolactone, (S)-massoia lactone, (Z)-dairy lactone, (Z)-gamma-jasmolactone, (Z)-yuzu lactone, (Z,E)-nepetalactone, 1beta-hydroxyalantolactone, 2-acetyl butyrolactone, 2-C-methyl-1,4-erythrono-D-lactone, 2-decen-1,4-lactone, 2-hydroxyalantolactone, 2-isobutyl-gamma-valerolactone, 23-hydroxyphysalolactone, 3-isobutenyl-5-methyl-5-vinyl butyrolactone, 3-methyl butyrolactone, 4-ethyl-gamma-octalactone, 4-hydroxy-3-(methylthio) butanoic acid lactone, 5-hydroxy-7-decenoic acid lactone, 9-hydroxy-gamma-tetradecalactone, alantolactone, alpha-angelica lactone, alpha-decalactone, alpha-methyl-gamma-butyrolactone, animal carbolactone, beta-angelica lactone, beta-methyl-delta-valerolactone, beta-propiolactone, blumealactone A, blumealactone B, blumealactone C, butyrolactonethiol, cassialactone, catalpalactone, cis, cis-nepetalactone, cis-oak lactone, costus lactone, costus valerolactone, creamy lactone, curcumalactone, D-mevalonolactone. dairy lactone, dehydrocostus lactone, dehydrodiferulic dilactone, dehydroindicolactone, delta-2-dodecenolactone, delta-decalactone, delta-dodecalactone, delta-gluconolactone, delta-heptalactone, delta-hexalactone, delta-juniper lactone, delta-nonalactone, delta-octadecalactone, delta-octalactone, or delta-tetradecalactone or a mixture thereof.

In another aspect the invention provides a method of modulating gene expression in tissues in a plant in need thereof, comprising contacting the plant with 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, methylglyoxal, benzyl nicotinate, a flavone, phenethyl resorcinol, acacetin, eriodictyol, phloretin, 2-phenylethyl 2-aminobenzoate, aromadedrin, benzoyl peroxide, heliotropyl phenyl acetate, chalcone, benzyl para-anisate, cinnamyl anthranilate, (Z)-2-penten-1-yl benzoate, 2-naphthyl phenyl ketone, bergamottin, isoimperatorin, para-cresyl benzoate, guaiacyl benzoate, caffeic acid ethyl ester, 4-phenyl propyl pyridine, 2-phenylethyl benzoate, benzyl benzoate, 2-methyl butyl salicylate, phenethyl sorbate, para-anisaldehyde, methyl anthranilate schiff's base, bibenzyl ether, ortho-anisyl benzoate, cinnamyl benzoate, 1-(2-hydroxy-4-methoxyphenyl)-3-(pyridin-2-yl) propan-1-one, ethyl 4-methylphenoxyacetate, benzyl trans-2-methyl-2-butenoate, alpha, alpha-dimethyl anisyl acetone, styryl acetate, amyl cinnamate, ortho-cresyl benzoate, methyl phenyl methylene aminobenzoate, phenethyl pyridine, (Z)-N-coumaroyl-5-hydroxyanthranilic acid, homoeriodictyol, benzyl 2,3-dimethyl-2-butenoate, meta-cresyl benzoate, (Z)-anisylidene acetone, guaiacyl phenyl acetate, 3-phenyl propyl salicylate, geranyl cantabiline, cortex pyridine, ethyl para-cresyl carbonate, ethyl phenoxyacetate, hesperetin, 3′,7-dihydroxy-4′-methoxyflavan, allyl (Z)-cinnamate, bisdemethoxycurcumin, amyl salicylate, phenyl acetaldehyde, methyl anthranilate schiff's base, p-tolyl phenylacetate, 4-formyl phenyl butyrate, 4-methoxybenzyl phenylacetate, para-cresyl 3-oxo-3-phenyl propionate, (E)-2-hexen-1-yl salicylate, furfuryl phenyl acetate, butyl 2-naphthyl ether, (E)-cinnamyl acetate, cinnamyl acetate, ethyl (E)-cinnamate, ethyl cinnamate, 2-para-cresyl oxyethyl acetate, neryl anthranilate, cinnamaldehyde, methyl anthranilate schiff's base, cinnamyl tiglate, (E)-cinnamyl propionate, 1-(4-Methoxyphenyl)-1-penten-3-one, imperatorin, para-cresyl valerate, meta-cresyl phenyl acetate, 3-methyl-3-buten-1-yl benzoate, para-anisyl acetoacetate, isoamyl p-methoxycinnamate, isoeugenyl benzoate, (Z)-3-hexen-1-yl cinnamate, eugenyl benzoate, isoamyl salicylate, phenylmethyl benzeneacetate, curcumin, butyl cinnamate, cinnamyl acetoacetate, para-cresyl phenyl ether, citral, methyl anthranilate schiff's base, cinnamyl valerate, xanthohumol, allyl phenoxyacetate, 2-phenylethyl 3-phenyl-2-propenoate, geranyl benzoate, (E)-3-butylidene phthalide, geranyl cinnamate, neryl cinnamate, cinnamyl cinnamate, amyl anthranilate, (E)-2-hexen-1-yl formate, (E)-3-hexen-1-yl formate, (E)-9-tetradecen-1-yl formate, (E)-cinnamyl formate, (E,Z)-2,6-nonadien-1-yl formate, (Z)-3-hexen-1-yl formate, (Z)-9-tetradecen-1-yl formate, 1,3,4,5,6,7-hexahydro-1,1,5,5-tetramethyl-2H-2,4a-methanonaphthalen-7-yl formate, 1-hexyl allyl formate, 2,6-dimethylhept-5-enyl formate, 2-decalinyl formate, 2-melozol formate, 2-methoxyethyl formate, 2-methyl butyl formate, 2-naphthyl formate, 2-octyl formate, 2-pentyl formate, 2-phenoxyethyl formate, 3-cyclohexyl 3-hexenyl formate, 3-mercapto-3-methyl butyl formate, 3-octyl formate, 3-phenyl propyl formate, 4-tert-amyl cyclohexyl formate, 4-tert-butyl cyclohexyl formate, allyl formate, alpha-amyl cinnamyl formate, alpha-campholenyl formate, aluminum triformate, amber formate, ammonium formate, amyl formate, benzyl formate, bornyl formate, butyl chloroformate, butyl formate, calcium formate, carvyl formate, caryophyllene formate, cedryl formate, chitosan formate, cholesteryl formate, cinnamyl formate, citronellyl formate, cuminyl formate, cyclodecyl formate, cyclododecyl formate, cyclohexyl formate, cyclooctyl formate, cymbopogon martini herb extract formate, decahydro-1,1,7-trimethyl-3a, 7-methano-3ah-cyclopentacyclooct-3-yl formate, decyl formate, dextro-bornyl formate, dihydrocarvyl formate, dihydrocitronellyl formate, dihydromyrcenyl formate, dimethyl benzyl carbinyl formate, dodecyl formate, ethyl 4-methyl-5-thiazolyl formate, ethyl benzoyl formate, ethyl chloroformate, ethyl formate, ethyl orthotrithioformate, ethyl vanillyl formate, eugenyl formate, fenchyl formate, furfuryl formate, geranyl formate, heliotropyl formate, heptyl formate, herbal cyclohexane formate, hexahydromethanoindenyl formate, hexahydrotetramethyl methanonaphthalene-8-methyl formate, hexyl formate, isoamyl formate, isobornyl formate, isobutyl formate, isodecyl formate, isoeugenyl formate, isononyl formate, isopropyl formate, isopulegyl formate, laevo-menthyl formate, linalyl formate, longifolene formate, marine formate, melozol formate, menthadienyl formate, menthyl formate, methyl formate, myristyl formate, myrtenyl formate, neryl formate, nonan-3-yl formate, nonisyl formate, nonyl formate, nopyl formate, octyl formate, oleyl formate, (±)-3-methyl-gamma-decalactone, (±)-chrysanthemolactone, (±)-dihydromint lactone, (±)-isoalantolactone, (±)-pantolactone, (±)-trans-whiskey lactone, (+/−)-mint lactone, (−)-cis-whiskey lactone, (−)-isomint lactone, (−)-mint lactone, (1R)-chrysanthemolactone, (1S)-chrysanthemolactone, (E)-dairy lactone, (R)-(−)-pantolactone, (R)-delta-decalactone, (R)-delta-dodecalactone, (R)-delta-hexalactone, (R)-delta-nonalactone, (R)-delta-octalactone, (R)-delta-undecalactone, (R)-epsilon-decalactone, (R)-gamma-decalactone, (R)-gamma-dodecalactone, (R)-gamma-heptalactone, (R)-gamma-hexalactone, (R)-gamma-nonalactone, (R)-gamma-octalactone, (R)-gamma-undecalactone, (R)-gamma-valerolactone, (R)-massoia lactone, (S)-delta-decalactone, (S)-delta-dodecalactone, (S)-delta-hexalactone, (S)-delta-nonalactone, (S)-delta-octalactone, (S)-delta-undecalactone, (S)-epsilon-decalactone, (S)-gamma-decalactone, (S)-gamma-dodecalactone, (S)-gamma-heptalactone, (S)-gamma-hexalactone, (S)-gamma-nonalactone, (S)-gamma-octalactone, (S)-gamma-undecalactone, (S)-gamma-valerolactone, (S)-massoia lactone, (Z)-dairy lactone, (Z)-gamma-jasmolactone, (Z)-yuzu lactone, (Z,E)-nepetalactone, 1beta-hydroxyalantolactone, 2-acetyl butyrolactone, 2-C-methyl-1,4-erythrono-D-lactone, 2-decen-1,4-lactone, 2-hydroxyalantolactone, 2-isobutyl-gamma-valerolactone, 23-hydroxyphysalolactone, 3-isobutenyl-5-methyl-5-vinyl butyrolactone, 3-methyl butyrolactone, 4-ethyl-gamma-octalactone, 4-hydroxy-3-(methylthio) butanoic acid lactone, 5-hydroxy-7-decenoic acid lactone, 9-hydroxy-gamma-tetradecalactone, alantolactone, alpha-angelica lactone, alpha-decalactone, alpha-methyl-gamma-butyrolactone, animal carbolactone, beta-angelica lactone, beta-methyl-delta-valerolactone, beta-propiolactone, blumealactone A, blumealactone B, blumealactone C, butyrolactonethiol, cassialactone, catalpalactone, cis, cis-nepetalactone, cis-oak lactone, costus lactone, costus valerolactone, creamy lactone, curcumalactone, D-mevalonolactone. dairy lactone, dehydrocostus lactone, dehydrodiferulic dilactone, dehydroindicolactone, delta-2-dodecenolactone, delta-decalactone, delta-dodecalactone, delta-gluconolactone, delta-heptalactone, delta-hexalactone, delta-juniper lactone, delta-nonalactone, delta-octadecalactone, delta-octalactone, or delta-tetradecalactone or a mixture thereof.

In another aspect the invention provides a method of treating or delaying the development of a cancer, treating or delaying the development of Alzheimer's disease, improving immune response, modulating gene expression in brain neurons, past the blood brain barrier, treating inflammation, or treating or delaying the development of chronic obstructive pulmonary disorder (COPD) in an animal in need thereof, comprising administering to the animal, an effective amount of 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, methylglyoxal, benzyl nicotinate, a flavone, phenethyl resorcinol, acacetin, eriodictyol, phloretin, 2-phenylethyl 2-aminobenzoate, aromadedrin, benzoyl peroxide, heliotropyl phenyl acetate, chalcone, benzyl para-anisate, cinnamyl anthranilate, (Z)-2-penten-1-yl benzoate, 2-naphthyl phenyl ketone, bergamottin, isoimperatorin, para-cresyl benzoate, guaiacyl benzoate, caffeic acid ethyl ester, 4-phenyl propyl pyridine, 2-phenylethyl benzoate, benzyl benzoate, 2-methyl butyl salicylate, phenethyl sorbate, para-anisaldehyde, methyl anthranilate schiff's base, bibenzyl ether, ortho-anisyl benzoate, cinnamyl benzoate, 1-(2-hydroxy-4-methoxyphenyl)-3-(pyridin-2-yl) propan-1-one, ethyl 4-methylphenoxyacetate, benzyl trans-2-methyl-2-butenoate, alpha,alpha-dimethyl anisyl acetone, styryl acetate, amyl cinnamate, ortho-cresyl benzoate, methyl phenyl methylene aminobenzoate, phenethyl pyridine, (Z)-N-coumaroyl-5-hydroxyanthranilic acid, homoeriodictyol, benzyl 2,3-dimethyl-2-butenoate, meta-cresyl benzoate, (Z)-anisylidene acetone, guaiacyl phenyl acetate, 3-phenyl propyl salicylate, geranyl cantabiline, cortex pyridine, ethyl para-cresyl carbonate, ethyl phenoxyacetate, hesperetin, 3′,7-dihydroxy-4′-methoxyflavan, allyl (Z)-cinnamate, bisdemethoxycurcumin, amyl salicylate, phenyl acetaldehyde, methyl anthranilate schiff's base, p-tolyl phenylacetate, 4-formyl phenyl butyrate, 4-methoxybenzyl phenylacetate, para-cresyl 3-oxo-3-phenyl propionate, (E)-2-hexen-1-yl salicylate, furfuryl phenyl acetate, butyl 2-naphthyl ether, (E)-cinnamyl acetate, cinnamyl acetate, ethyl (E)-cinnamate, ethyl cinnamate, 2-para-cresyl oxyethyl acetate, neryl anthranilate, cinnamaldehyde, methyl anthranilate schiff's base, cinnamyl tiglate, (E)-cinnamyl propionate, 1-(4-Methoxyphenyl)-1-penten-3-one, imperatorin, para-cresyl valerate, meta-cresyl phenyl acetate, 3-methyl-3-buten-1-yl benzoate, para-anisyl acetoacetate, isoamyl p-methoxycinnamate, isoeugenyl benzoate, (Z)-3-hexen-1-yl cinnamate, eugenyl benzoate, isoamyl salicylate, phenylmethyl benzeneacetate, curcumin, butyl cinnamate, cinnamyl acetoacetate, para-cresyl phenyl ether, citral, methyl anthranilate schiff's base, cinnamyl valerate, xanthohumol, allyl phenoxyacetate, 2-phenylethyl 3-phenyl-2-propenoate, geranyl benzoate, (E)-3-butylidene phthalide, geranyl cinnamate, neryl cinnamate, cinnamyl cinnamate, amyl anthranilate, (E)-2-hexen-1-yl formate, (E)-3-hexen-1-yl formate, (E)-9-tetradecen-1-yl formate, (E)-cinnamyl formate, (E,Z)-2,6-nonadien-1-yl formate, (Z)-3-hexen-1-yl formate, (Z)-9-tetradecen-1-yl formate, 1,3,4,5,6,7-hexahydro-1,1,5,5-tetramethyl-2H-2,4a-methanonaphthalen-7-yl formate, 1-hexyl allyl formate, 2,6-dimethylhept-5-enyl formate, 2-decalinyl formate, 2-melozol formate, 2-methoxyethyl formate, 2-methyl butyl formate, 2-naphthyl formate, 2-octyl formate, 2-pentyl formate, 2-phenoxyethyl formate, 3-cyclohexyl 3-hexenyl formate, 3-mercapto-3-methyl butyl formate, 3-octyl formate, 3-phenyl propyl formate, 4-tert-amyl cyclohexyl formate, 4-tert-butyl cyclohexyl formate, allyl formate, alpha-amyl cinnamyl formate, alpha-campholenyl formate, aluminum triformate, amber formate, ammonium formate, amyl formate, benzyl formate, bornyl formate, butyl chloroformate, butyl formate, calcium formate, carvyl formate, caryophyllene formate, cedryl formate, chitosan formate, cholesteryl formate, cinnamyl formate, citronellyl formate, cuminyl formate, cyclodecyl formate, cyclododecyl formate, cyclohexyl formate, cyclooctyl formate, cymbopogon martini herb extract formate, decahydro-1,1,7-trimethyl-3a,7-methano-3ah-cyclopentacyclooct-3-yl formate, decyl formate, dextro-bornyl formate, dihydrocarvyl formate, dihydrocitronellyl formate, dihydromyrcenyl formate, dimethyl benzyl carbinyl formate, dodecyl formate, ethyl 4-methyl-5-thiazolyl formate, ethyl benzoyl formate, ethyl chloroformate, ethyl formate, ethyl orthotrithioformate, ethyl vanillyl formate, eugenyl formate, fenchyl formate, furfuryl formate, geranyl formate, heliotropyl formate, heptyl formate, herbal cyclohexane formate, hexahydromethanoindenyl formate, hexahydrotetramethyl methanonaphthalene-8-methyl formate, hexyl formate, isoamyl formate, isobornyl formate, isobutyl formate, isodecyl formate, isoeugenyl formate, isononyl formate, isopropyl formate, isopulegyl formate, laevo-menthyl formate, linalyl formate, longifolene formate, marine formate, melozol formate, menthadienyl formate, menthyl formate, methyl formate, myristyl formate, myrtenyl formate, neryl formate, nonan-3-yl formate, nonisyl formate, nonyl formate, nopyl formate, octyl formate, oleyl formate, (±)-3-methyl-gamma-decalactone, (±)-chrysanthemolactone, (±)-dihydromint lactone, (±)-isoalantolactone, (±)-pantolactone, (±)-trans-whiskey lactone, (+/−)-mint lactone, (−)-cis-whiskey lactone, (−)-isomint lactone, (−)-mint lactone, (1R)-chrysanthemolactone, (1S)-chrysanthemolactone, (E)-dairy lactone, (R)-(−)-pantolactone, (R)-delta-decalactone, (R)-delta-dodecalactone, (R)-delta-hexalactone, (R)-delta-nonalactone, (R)-delta-octalactone, (R)-delta-undecalactone, (R)-epsilon-decalactone, (R)-gamma-decalactone, (R)-gamma-dodecalactone, (R)-gamma-heptalactone, (R)-gamma-hexalactone, (R)-gamma-nonalactone, (R)-gamma-octalactone, (R)-gamma-undecalactone, (R)-gamma-valerolactone, (R)-massoia lactone, (S)-delta-decalactone, (S)-delta-dodecalactone, (S)-delta-hexalactone, (S)-delta-nonalactone, (S)-delta-octalactone, (S)-delta-undecalactone, (S)-epsilon-decalactone, (S)-gamma-decalactone, (S)-gamma-dodecalactone, (S)-gamma-heptalactone, (S)-gamma-hexalactone, (S)-gamma-nonalactone, (S)-gamma-octalactone, (S)-gamma-undecalactone, (S)-gamma-valerolactone, (S)-massoia lactone, (Z)-dairy lactone, (Z)-gamma-jasmolactone, (Z)-yuzu lactone, (Z,E)-nepetalactone, 1beta-hydroxyalantolactone, 2-acetyl butyrolactone, 2-C-methyl-1,4-erythrono-D-lactone, 2-decen-1,4-lactone, 2-hydroxyalantolactone, 2-isobutyl-gamma-valerolactone, 23-hydroxyphysalolactone, 3-isobutenyl-5-methyl-5-vinyl butyrolactone, 3-methyl butyrolactone, 4-ethyl-gamma-octalactone, 4-hydroxy-3-(methylthio) butanoic acid lactone, 5-hydroxy-7-decenoic acid lactone, 9-hydroxy-gamma-tetradecalactone, alantolactone, alpha-angelica lactone, alpha-decalactone, alpha-methyl-gamma-butyrolactone, animal carbolactone, beta-angelica lactone, beta-methyl-delta-valerolactone, beta-propiolactone, blumealactone A, blumealactone B, blumealactone C, butyrolactonethiol, cassialactone, catalpalactone, cis, cis-nepetalactone, cis-oak lactone, costus lactone, costus valerolactone, creamy lactone, curcumalactone, D-mevalonolactone. dairy lactone, dehydrocostus lactone, dehydrodiferulic dilactone, dehydroindicolactone, delta-2-dodecenolactone, delta-decalactone, delta-dodecalactone, delta-gluconolactone, delta-heptalactone, delta-hexalactone, delta-juniper lactone, delta-nonalactone, delta-octadecalactone, delta-octalactone, or delta-tetradecalactone or a mixture thereof.

In another aspect the invention provides a pharmaceutical composition, comprising 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, methylglyoxal, benzyl nicotinate, a flavone, phenethyl resorcinol, acacetin, eriodictyol, phloretin, 2-phenylethyl 2-aminobenzoate, aromadedrin, benzoyl peroxide, heliotropyl phenyl acetate, chalcone, benzyl para-anisate, cinnamyl anthranilate, (Z)-2-penten-1-yl benzoate, 2-naphthyl phenyl ketone, bergamottin, isoimperatorin, para-cresyl benzoate, guaiacyl benzoate, caffeic acid ethyl ester, 4-phenyl propyl pyridine, 2-phenylethyl benzoate, benzyl benzoate, 2-methyl butyl salicylate, phenethyl sorbate, para-anisaldehyde, methyl anthranilate schiff's base, bibenzyl ether, ortho-anisyl benzoate, cinnamyl benzoate, 1-(2-hydroxy-4-methoxyphenyl)-3-(pyridin-2-yl) propan-1-one, ethyl 4-methylphenoxyacetate, benzyl trans-2-methyl-2-butenoate, alpha, alpha-dimethyl anisyl acetone, styryl acetate, amyl cinnamate, ortho-cresyl benzoate, methyl phenyl methylene aminobenzoate, phenethyl pyridine, (Z)-N-coumaroyl-5-hydroxyanthranilic acid, homoeriodictyol, benzyl 2,3-dimethyl-2-butenoate, meta-cresyl benzoate, (Z)-anisylidene acetone, guaiacyl phenyl acetate, 3-phenyl propyl salicylate, geranyl cantabiline, cortex pyridine, ethyl para-cresyl carbonate, ethyl phenoxyacetate, hesperetin, 3′,7-dihydroxy-4′-methoxyflavan, allyl (Z)-cinnamate, bisdemethoxycurcumin, amyl salicylate, phenyl acetaldehyde, methyl anthranilate schiff's base, p-tolyl phenylacetate, 4-formyl phenyl butyrate, 4-methoxybenzyl phenylacetate, para-cresyl 3-oxo-3-phenyl propionate, (E)-2-hexen-1-yl salicylate, furfuryl phenyl acetate, butyl 2-naphthyl ether, (E)-cinnamyl acetate, cinnamyl acetate, ethyl (E)-cinnamate, ethyl cinnamate, 2-para-cresyl oxyethyl acetate, neryl anthranilate, cinnamaldehyde, methyl anthranilate schiff's base, cinnamyl tiglate, (E)-cinnamyl propionate, 1-(4-Methoxyphenyl)-1-penten-3-one, imperatorin, para-cresyl valerate, meta-cresyl phenyl acetate, 3-methyl-3-buten-1-yl benzoate, para-anisyl acetoacetate, isoamyl p-methoxycinnamate, isoeugenyl benzoate, (Z)-3-hexen-1-yl cinnamate, eugenyl benzoate, isoamyl salicylate, phenylmethyl benzeneacetate, curcumin, butyl cinnamate, cinnamyl acetoacetate, para-cresyl phenyl ether, citral, methyl anthranilate schiff's base, cinnamyl valerate, xanthohumol, allyl phenoxyacetate, 2-phenylethyl 3-phenyl-2-propenoate, geranyl benzoate, (E)-3-butylidene phthalide, geranyl cinnamate, neryl cinnamate, cinnamyl cinnamate, amyl anthranilate, (E)-2-hexen-1-yl formate, (E)-3-hexen-1-yl formate, (E)-9-tetradecen-1-yl formate, (E)-cinnamyl formate, (E,Z)-2,6-nonadien-1-yl formate, (Z)-3-hexen-1-yl formate, (Z)-9-tetradecen-1-yl formate, 1,3,4,5,6,7-hexahydro-1,1,5,5-tetramethyl-2H-2,4a-methanonaphthalen-7-yl formate, 1-hexyl allyl formate, 2,6-dimethylhept-5-enyl formate, 2-decalinyl formate, 2-melozol formate, 2-methoxyethyl formate, 2-methyl butyl formate, 2-naphthyl formate, 2-octyl formate, 2-pentyl formate, 2-phenoxyethyl formate, 3-cyclohexyl 3-hexenyl formate, 3-mercapto-3-methyl butyl formate, 3-octyl formate, 3-phenyl propyl formate, 4-tert-amyl cyclohexyl formate, 4-tert-butyl cyclohexyl formate, allyl formate, alpha-amyl cinnamyl formate, alpha-campholenyl formate, aluminum triformate, amber formate, ammonium formate, amyl formate, benzyl formate, bornyl formate, butyl chloroformate, butyl formate, calcium formate, carvyl formate, caryophyllene formate, cedryl formate, chitosan formate, cholesteryl formate, cinnamyl formate, citronellyl formate, cuminyl formate, cyclodecyl formate, cyclododecyl formate, cyclohexyl formate, cyclooctyl formate, cymbopogon martini herb extract formate, decahydro-1,1,7-trimethyl-3a,7-methano-3ah-cyclopentacyclooct-3-yl formate, decyl formate, dextro-bornyl formate, dihydrocarvyl formate, dihydrocitronellyl formate, dihydromyrcenyl formate, dimethyl benzyl carbinyl formate, dodecyl formate, ethyl 4-methyl-5-thiazolyl formate, ethyl benzoyl formate, ethyl chloroformate, ethyl formate, ethyl orthotrithioformate, ethyl vanillyl formate, eugenyl formate, fenchyl formate, furfuryl formate, geranyl formate, heliotropyl formate, heptyl formate, herbal cyclohexane formate, hexahydromethanoindenyl formate, hexahydrotetramethyl methanonaphthalene-8-methyl formate, hexyl formate, isoamyl formate, isobornyl formate, isobutyl formate, isodecyl formate, isoeugenyl formate, isononyl formate, isopropyl formate, isopulegyl formate, laevo-menthyl formate, linalyl formate, longifolene formate, marine formate, melozol formate, menthadienyl formate, menthyl formate, methyl formate, myristyl formate, myrtenyl formate, neryl formate, nonan-3-yl formate, nonisyl formate, nonyl formate, nopyl formate, octyl formate, oleyl formate, (±)-3-methyl-gamma-decalactone, (±)-chrysanthemolactone, (±)-dihydromint lactone, (±)-isoalantolactone, (±)-pantolactone, (±)-trans-whiskey lactone, (+/−)-mint lactone, (−)-cis-whiskey lactone, (−)-isomint lactone, (−)-mint lactone, (1R)-chrysanthemolactone, (1S)-chrysanthemolactone, (E)-dairy lactone, (R)-(−)-pantolactone, (R)-delta-decalactone, (R)-delta-dodecalactone, (R)-delta-hexalactone, (R)-delta-nonalactone, (R)-delta-octalactone, (R)-delta-undecalactone, (R)-epsilon-decalactone, (R)-gamma-decalactone, (R)-gamma-dodecalactone, (R)-gamma-heptalactone, (R)-gamma-hexalactone, (R)-gamma-nonalactone, (R)-gamma-octalactone, (R)-gamma-undecalactone, (R)-gamma-valerolactone, (R)-massoia lactone, (S)-delta-decalactone, (S)-delta-dodecalactone, (S)-delta-hexalactone, (S)-delta-nonalactone, (S)-delta-octalactone, (S)-delta-undecalactone, (S)-epsilon-decalactone, (S)-gamma-decalactone, (S)-gamma-dodecalactone, (S)-gamma-heptalactone, (S)-gamma-hexalactone, (S)-gamma-nonalactone, (S)-gamma-octalactone, (S)-gamma-undecalactone, (S)-gamma-valerolactone, (S)-massoia lactone, (Z)-dairy lactone, (Z)-gamma-jasmolactone, (Z)-yuzu lactone, (Z,E)-nepetalactone, 1beta-hydroxyalantolactone, 2-acetyl butyrolactone, 2-C-methyl-1,4-erythrono-D-lactone, 2-decen-1,4-lactone, 2-hydroxyalantolactone, 2-isobutyl-gamma-valerolactone, 23-hydroxyphysalolactone, 3-isobutenyl-5-methyl-5-vinyl butyrolactone, 3-methyl butyrolactone, 4-ethyl-gamma-octalactone, 4-hydroxy-3-(methylthio) butanoic acid lactone, 5-hydroxy-7-decenoic acid lactone, 9-hydroxy-gamma-tetradecalactone, alantolactone, alpha-angelica lactone, alpha-decalactone, alpha-methyl-gamma-butyrolactone, animal carbolactone, beta-angelica lactone, beta-methyl-delta-valerolactone, beta-propiolactone, blumealactone A, blumealactone B, blumealactone C, butyrolactonethiol, cassialactone, catalpalactone, cis,cis-nepetalactone, cis-oak lactone, costus lactone, costus valerolactone, creamy lactone, curcumalactone, D-mevalonolactone. dairy lactone, dehydrocostus lactone, dehydrodiferulic dilactone, dehydroindicolactone, delta-2-dodecenolactone, delta-decalactone, delta-dodecalactone, delta-gluconolactone, delta-heptalactone, delta-hexalactone, delta-juniper lactone, delta-nonalactone, delta-octadecalactone, delta-octalactone, or delta-tetradecalactone; and a pharmaceutically acceptable excipient.

In another aspect the invention provides a kit, comprising:

a compound selected from the group consisting of 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, methylglyoxal, benzyl nicotinate, a flavone, phenethyl resorcinol, acacetin, eriodictyol, phloretin, 2-phenylethyl 2-aminobenzoate, aromadedrin, benzoyl peroxide, heliotropyl phenyl acetate, chalcone, benzyl para-anisate, cinnamyl anthranilate, (Z)-2-penten-1-yl benzoate, 2-naphthyl phenyl ketone, bergamottin, isoimperatorin, para-cresyl benzoate, guaiacyl benzoate, caffeic acid ethyl ester, 4-phenyl propyl pyridine, 2-phenylethyl benzoate, benzyl benzoate, 2-methyl butyl salicylate, phenethyl sorbate, para-anisaldehyde, methyl anthranilate schiff's base, bibenzyl ether, ortho-anisyl benzoate, cinnamyl benzoate, 1-(2-hydroxy-4-methoxyphenyl)-3-(pyridin-2-yl) propan-1-one, ethyl 4-methylphenoxyacetate, benzyl trans-2-methyl-2-butenoate, alpha, alpha-dimethyl anisyl acetone, styryl acetate, amyl cinnamate, ortho-cresyl benzoate, methyl phenyl methylene aminobenzoate, phenethyl pyridine, (Z)-N-coumaroyl-5-hydroxyanthranilic acid, homoeriodictyol, benzyl 2,3-dimethyl-2-butenoate, meta-cresyl benzoate, (Z)-anisylidene acetone, guaiacyl phenyl acetate, 3-phenyl propyl salicylate, geranyl cantabiline, cortex pyridine, ethyl para-cresyl carbonate, ethyl phenoxyacetate, hesperetin, 3′,7-dihydroxy-4′-methoxyflavan, allyl (Z)-cinnamate, bisdemethoxycurcumin, amyl salicylate, phenyl acetaldehyde, methyl anthranilate schiff's base, p-tolyl phenylacetate, 4-formyl phenyl butyrate, 4-methoxybenzyl phenylacetate, para-cresyl 3-oxo-3-phenyl propionate, (E)-2-hexen-1-yl salicylate, furfuryl phenyl acetate, butyl 2-naphthyl ether, (E)-cinnamyl acetate, cinnamyl acetate, ethyl (E)-cinnamate, ethyl cinnamate, 2-para-cresyl oxyethyl acetate, neryl anthranilate, cinnamaldehyde, methyl anthranilate schiff's base, cinnamyl tiglate, (E)-cinnamyl propionate, 1-(4-Methoxyphenyl)-1-penten-3-one, imperatorin, para-cresyl valerate, meta-cresyl phenyl acetate, 3-methyl-3-buten-1-yl benzoate, para-anisyl acetoacetate, isoamyl p-methoxycinnamate, isoeugenyl benzoate, (Z)-3-hexen-1-yl cinnamate, eugenyl benzoate, isoamyl salicylate, phenylmethyl benzeneacetate, curcumin, butyl cinnamate, cinnamyl acetoacetate, para-cresyl phenyl ether, citral, methyl anthranilate schiff's base, cinnamyl valerate, xanthohumol, allyl phenoxyacetate, 2-phenylethyl 3-phenyl-2-propenoate, geranyl benzoate, (E)-3-butylidene phthalide, geranyl cinnamate, neryl cinnamate, cinnamyl cinnamate, amyl anthranilate, (E)-2-hexen-1-yl formate, (E)-3-hexen-1-yl formate, (E)-9-tetradecen-1-yl formate, (E)-cinnamyl formate, (E,Z)-2,6-nonadien-1-yl formate, (Z)-3-hexen-1-yl formate, (Z)-9-tetradecen-1-yl formate, 1,3,4,5,6,7-hexahydro-1,1,5,5-tetramethyl-2H-2,4a-methanonaphthalen-7-yl formate, 1-hexyl allyl formate, 2,6-dimethylhept-5-enyl formate, 2-decalinyl formate, 2-melozol formate, 2-methoxyethyl formate, 2-methyl butyl formate, 2-naphthyl formate, 2-octyl formate, 2-pentyl formate, 2-phenoxyethyl formate, 3-cyclohexyl 3-hexenyl formate, 3-mercapto-3-methyl butyl formate, 3-octyl formate, 3-phenyl propyl formate, 4-tert-amyl cyclohexyl formate, 4-tert-butyl cyclohexyl formate, allyl formate, alpha-amyl cinnamyl formate, alpha-campholenyl formate, aluminum triformate, amber formate, ammonium formate, amyl formate, benzyl formate, bornyl formate, butyl chloroformate, butyl formate, calcium formate, carvyl formate, caryophyllene formate, cedryl formate, chitosan formate, cholesteryl formate, cinnamyl formate, citronellyl formate, cuminyl formate, cyclodecyl formate, cyclododecyl formate, cyclohexyl formate, cyclooctyl formate, cymbopogon martini herb extract formate, decahydro-1,1,7-trimethyl-3a, 7-methano-3ah-cyclopentacyclooct-3-yl formate, decyl formate, dextro-bornyl formate, dihydrocarvyl formate, dihydrocitronellyl formate, dihydromyrcenyl formate, dimethyl benzyl carbinyl formate, dodecyl formate, ethyl 4-methyl-5-thiazolyl formate, ethyl benzoyl formate, ethyl chloroformate, ethyl formate, ethyl orthotrithioformate, ethyl vanillyl formate, eugenyl formate, fenchyl formate, furfuryl formate, geranyl formate, heliotropyl formate, heptyl formate, herbal cyclohexane formate, hexahydromethanoindenyl formate, hexahydrotetramethyl methanonaphthalene-8-methyl formate, hexyl formate, isoamyl formate, isobornyl formate, isobutyl formate, isodecyl formate, isoeugenyl formate, isononyl formate, isopropyl formate, isopulegyl formate, laevo-menthyl formate, linalyl formate, longifolene formate, marine formate, melozol formate, menthadienyl formate, menthyl formate, methyl formate, myristyl formate, myrtenyl formate, neryl formate, nonan-3-yl formate, nonisyl formate, nonyl formate, nopyl formate, octyl formate, oleyl formate, (±)-3-methyl-gamma-decalactone, (±)-chrysanthemolactone, (±)-dihydromint lactone, (±)-isoalantolactone, (±)-pantolactone, (±)-trans-whiskey lactone, (+/−)-mint lactone, (−)-cis-whiskey lactone, (−)-isomint lactone, (−)-mint lactone, (1R)-chrysanthemolactone, (1S)-chrysanthemolactone, (E)-dairy lactone, (R)-(−)-pantolactone, (R)-delta-decalactone, (R)-delta-dodecalactone, (R)-delta-hexalactone, (R)-delta-nonalactone, (R)-delta-octalactone, (R)-delta-undecalactone, (R)-epsilon-decalactone, (R)-gamma-decalactone, (R)-gamma-dodecalactone, (R)-gamma-heptalactone, (R)-gamma-hexalactone, (R)-gamma-nonalactone, (R)-gamma-octalactone, (R)-gamma-undecalactone, (R)-gamma-valerolactone, (R)-massoia lactone, (S)-delta-decalactone, (S)-delta-dodecalactone, (S)-delta-hexalactone, (S)-delta-nonalactone, (S)-delta-octalactone, (S)-delta-undecalactone, (S)-epsilon-decalactone, (S)-gamma-decalactone, (S)-gamma-dodecalactone, (S)-gamma-heptalactone, (S)-gamma-hexalactone, (S)-gamma-nonalactone, (S)-gamma-octalactone, (S)-gamma-undecalactone, (S)-gamma-valerolactone, (S)-massoia lactone, (Z)-dairy lactone, (Z)-gamma-jasmolactone, (Z)-yuzu lactone, (Z,E)-nepetalactone, 1beta-hydroxyalantolactone, 2-acetyl butyrolactone, 2-C-methyl-1,4-erythrono-D-lactone, 2-decen-1,4-lactone, 2-hydroxyalantolactone, 2-isobutyl-gamma-valerolactone, 23-hydroxyphysalolactone, 3-isobutenyl-5-methyl-5-vinyl butyrolactone, 3-methyl butyrolactone, 4-ethyl-gamma-octalactone, 4-hydroxy-3-(methylthio) butanoic acid lactone, 5-hydroxy-7-decenoic acid lactone, 9-hydroxy-gamma-tetradecalactone, alantolactone, alpha-angelica lactone, alpha-decalactone, alpha-methyl-gamma-butyrolactone, animal carbolactone, beta-angelica lactone, beta-methyl-delta-valerolactone, beta-propiolactone, blumealactone A, blumealactone B, blumealactone C, butyrolactonethiol, cassialactone, catalpalactone, cis,cis-nepetalactone, cis-oak lactone, costus lactone, costus valerolactone, creamy lactone, curcumalactone, D-mevalonolactone. dairy lactone, dehydrocostus lactone, dehydrodiferulic dilactone, dehydroindicolactone, delta-2-dodecenolactone, delta-decalactone, delta-dodecalactone, delta-gluconolactone, delta-heptalactone, delta-hexalactone, delta-juniper lactone, delta-nonalactone, delta-octadecalactone, delta-octalactone, and delta-tetradecalactone; and
a label and/or instructions for use of the compounds in treatment of a neurological disorder, cancer, chronic obstructive pulmonary disorder (COPD), or an infectious disease.

In another aspect the invention provides an article of manufacture, comprising:
a compound selected from the group consisting of 3,4-hexanedione, methyl pyruvate ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, methylglyoxal, benzyl nicotinate, a flavone, phenethyl resorcinol, acacetin, eriodictyol, phloretin, 2-phenylethyl 2-aminobenzoate, aromadedrin, benzoyl peroxide, heliotropyl phenyl acetate, chalcone, benzyl para-anisate, cinnamyl anthranilate, (Z)-2-penten-1-yl benzoate, 2-naphthyl phenyl ketone, bergamottin, isoimperatorin, para-cresyl benzoate, guaiacyl benzoate, caffeic acid ethyl ester, 4-phenyl propyl pyridine, 2-phenylethyl benzoate, benzyl benzoate, 2-methyl butyl salicylate, phenethyl sorbate, para-anisaldehyde, methyl anthranilate schiff's base, bibenzyl ether, ortho-anisyl benzoate, cinnamyl benzoate, 1-(2-hydroxy-4-methoxyphenyl)-3-(pyridin-2-yl) propan-1-one, ethyl 4-methylphenoxyacetate, benzyl trans-2-methyl-2-butenoate, alpha, alpha-dimethyl anisyl acetone, styryl acetate, amyl cinnamate, ortho-cresyl benzoate, methyl phenyl methylene aminobenzoate, phenethyl pyridine, (Z)-N-coumaroyl-5-hydroxyanthranilic acid, homoeriodictyol, benzyl 2,3-dimethyl-2-butenoate, meta-cresyl benzoate, (Z)-anisylidene acetone, guaiacyl phenyl acetate, 3-phenyl propyl salicylate, geranyl cantabiline, cortex pyridine, ethyl para-cresyl carbonate, ethyl phenoxyacetate, hesperetin, 3′,7-dihydroxy-4′-methoxyflavan, allyl (Z)-cinnamate, bisdemethoxycurcumin, amyl salicylate, phenyl acetaldehyde, methyl anthranilate schiff's base, p-tolyl phenylacetate, 4-formyl phenyl butyrate, 4-methoxybenzyl phenylacetate, para-cresyl 3-oxo-3-phenyl propionate, (E)-2-hexen-1-yl salicylate, furfuryl phenyl acetate, butyl 2-naphthyl ether, (E)-cinnamyl acetate, cinnamyl acetate, ethyl (E)-cinnamate, ethyl cinnamate, 2-para-cresyl oxyethyl acetate, neryl anthranilate, cinnamaldehyde, methyl anthranilate schiff's base, cinnamyl tiglate, (E)-cinnamyl propionate, 1-(4-Methoxyphenyl)-1-penten-3-one, imperatorin, para-cresyl valerate, meta-cresyl phenyl acetate, 3-methyl-3-buten-1-yl benzoate, para-anisyl acetoacetate, isoamyl p-methoxycinnamate, isoeugenyl benzoate, (Z)-3-hexen-1-yl cinnamate, eugenyl benzoate, isoamyl salicylate, phenylmethyl benzeneacetate, curcumin, butyl cinnamate, cinnamyl acetoacetate, para-cresyl phenyl ether, citral, methyl anthranilate schiff's base, cinnamyl valerate, xanthohumol, allyl phenoxyacetate, 2-phenylethyl 3-phenyl-2-propenoate, geranyl benzoate, (E)-3-butylidene phthalide, geranyl cinnamate, neryl cinnamate, cinnamyl cinnamate, amyl anthranilate, (E)-2-hexen-1-yl formate, (E)-3-hexen-1-yl formate, (E)-9-tetradecen-1-yl formate, (E)-cinnamyl formate, (E,Z)-2,6-nonadien-1-yl formate, (Z)-3-hexen-1-yl formate, (Z)-9-tetradecen-1-yl formate, 1,3,4,5,6,7-hexahydro-1,1,5,5-tetramethyl-2H-2,4a-methanonaphthalen-7-yl formate, 1-hexyl allyl formate, 2,6-dimethylhept-5-enyl formate, 2-decalinyl formate, 2-melozol formate, 2-methoxyethyl formate, 2-methyl butyl formate, 2-naphthyl formate, 2-octyl formate, 2-pentyl formate, 2-phenoxyethyl formate, 3-cyclohexyl 3-hexenyl formate, 3-mercapto-3-methyl butyl formate, 3-octyl formate, 3-phenyl propyl formate, 4-tert-amyl cyclohexyl formate, 4-tert-butyl cyclohexyl formate, allyl formate, alpha-amyl cinnamyl formate, alpha-campholenyl formate, aluminum triformate, amber formate, ammonium formate, amyl formate, benzyl formate, bornyl formate, butyl chloroformate, butyl formate, calcium formate, carvyl formate, caryophyllene formate, cedryl formate, chitosan formate, cholesteryl formate, cinnamyl formate, citronellyl formate, cuminyl formate, cyclodecyl formate, cyclododecyl formate, cyclohexyl formate, cyclooctyl formate, cymbopogon martini herb extract formate, decahydro-1,1,7-trimethyl-3a, 7-methano-3ah-cyclopentacyclooct-3-yl formate, decyl formate, dextro-bornyl formate, dihydrocarvyl formate, dihydrocitronellyl formate, dihydromyrcenyl formate, dimethyl benzyl carbinyl formate, dodecyl formate, ethyl 4-methyl-5-thiazolyl formate, ethyl benzoyl formate, ethyl chloroformate, ethyl formate, ethyl orthotrithioformate, ethyl vanillyl formate, eugenyl formate, fenchyl formate, furfuryl formate, geranyl formate, heliotropyl formate, heptyl formate, herbal cyclohexane formate, hexahydromethanoindenyl formate, hexahydrotetramethyl methanonaphthalene-8-methyl formate, hexyl formate, isoamyl formate, isobornyl formate, isobutyl formate, isodecyl formate, isoeugenyl formate, isononyl formate, isopropyl formate, isopulegyl formate, laevo-menthyl formate, linalyl formate, longifolene formate, marine formate, melozol formate, menthadienyl formate, menthyl formate, methyl formate, myristyl formate, myrtenyl formate, neryl formate, nonan-3-yl formate, nonisyl formate, nonyl formate, nopyl formate, octyl formate, oleyl formate, (±)-3-methyl-gamma-decalactone, (±)-chrysanthemolactone, (±)-dihydromint lactone, (±)-isoalantolactone, (±)-pantolactone, (±)-trans-whiskey lactone, (+/−)-mint lactone, (−)-cis-whiskey lactone, (−)-isomint lactone, (−)-mint lactone, (1R)-chrysanthemolactone, (1S)-chrysanthemolactone, (E)-dairy lactone, (R)-(−)-pantolactone, (R)-delta-decalactone, (R)-delta-dodecalactone, (R)-delta-hexalactone, (R)-delta-nonalactone, (R)-delta-octalactone, (R)-delta-undecalactone, (R)-epsilon-decalactone, (R)-gamma-decalactone, (R)-gamma-dodecalactone, (R)-gamma-heptalactone, (R)-gamma-hexalactone, (R)-gamma-nonalactone, (R)-gamma-octalactone, (R)-gamma-undecalactone, (R)-gamma-valerolactone, (R)-massoia lactone, (S)-delta-decalactone, (S)-delta-dodecalactone, (S)-delta-hexalactone, (S)-delta-nonalactone, (S)-delta-octalactone, (S)-delta-undecalactone, (S)-epsilon-decalactone, (S)-gamma-decalactone, (S)-gamma-dodecalactone, (S)-gamma-heptalactone, (S)-gamma-hexalactone, (S)-gamma-nonalactone, (S)-gamma-octalactone, (S)-gamma-undecalactone, (S)-gamma-valerolactone, (S)-massoia lactone, (Z)-dairy lactone, (Z)-gamma-jasmolactone, (Z)-yuzu lactone, (Z,E)-nepetalactone, 1beta-hydroxyalantolactone, 2-acetyl butyrolactone, 2-C-methyl-1,4-erythrono-D-lactone, 2-decen-1,4-lactone, 2-hydroxyalantolactone, 2-isobutyl-gamma-valerolactone, 23-hydroxyphysalolactone, 3-isobutenyl-5-methyl-5-vinyl butyrolactone, 3-methyl butyrolactone, 4-ethyl-gamma-octalactone, 4-hydroxy-3-(methylthio) butanoic acid lactone, 5-hydroxy-7-decenoic acid lactone, 9-hydroxy-gamma-tetradecalactone, alantolactone, alpha-angelica lactone, alpha-decalactone, alpha-methyl-gamma-butyrolactone, animal carbolactone, beta-angelica lactone, beta-methyl-delta-valerolactone, beta-propiolactone, blumealactone A, blumealactone B, blumealactone C, butyrolactonethiol, cassialactone, catalpalactone, cis,cis-nepetalactone, cis-oak lactone, costus lactone, costus valerolactone, creamy lactone, curcumalactone, D-mevalonolactone. dairy lactone, dehydrocostus lactone, dehydrodiferulic dilactone, dehydroindicolactone, delta-2-dodecenolactone, delta-decalactone, delta-dodecalactone, delta-gluconolactone, delta-heptalactone, delta-hexalactone, delta-juniper lactone, delta-nonalactone, delta-octadecalactone, delta-octalactone, and delta-tetradecalactone; and
a suitable container (e.g., a suitable dispenser).

The invention also provides a compound as described herein for use in medical therapy.

The invention also provides a compound as described herein for treating or delaying the development of a cancer, treating or delaying the development of Alzheimer's disease, improving immune response, modulating gene expression in brain neurons, past the blood brain barrier, treating inflammation, or treating or delaying the development of chronic obstructive pulmonary disorder (COPD).

The invention also provides the use of a compound of formula I or a pharmaceutically acceptable salt thereof to prepare a medicament for treating or delaying the development of a cancer, treating or delaying the development of Alzheimer's disease, improving immune response, modulating gene expression in brain neurons, past the blood brain barrier, treating inflammation, or treating or delaying the development of chronic obstructive pulmonary disorder (COPD) in an animal (e.g. a mammal such as a human).

The invention also provides a method comprising: 1) altering or delaying development of plant tissue, 2) altering or delaying response to abiotic stress, or infection, in a plant, or 3) modulating gene expression in tissues in a plant, comprising contacting the plant with an HDAC inhibitor.

The invention also provides the use of an HDAC inhibitor to: 1) alter or delay development of plant tissue, 2) alter or delay response to abiotic stress, or infection, in a plant, or 3) modulate gene expression in tissues in a plant.

Many of the HDAC inhibiting compounds listed above have very low toxicity, are natural, have widespread use in flavors, fragrances, are considered GRAS (Generally regarded as safe) by the FDA and FEMA (Flavors and Extracts Manufacturers Association), extremely affordable (<420/kg) and therefore raise the potential for use in a novel inhaled therapeutic approach.

### BRIEF DESCRIPTION OF THE DRAWINGS

FIG. 1. Class I HDAC3% Inhibition Results: 12 compounds, including 2,3-butanedione, were screened for HDAC 3 inhibition.

FIG. 2. Class IIa HDAC4% Inhibition Results: 12 compounds, including 2,3-butanedione, were screened for HDAC 4 inhibition.

FIG. 3. Class IIb HDAC6% Inhibition Results: 27 compounds, including 2,3-butanedione, were screened for HDAC 6 inhibition.

FIG. 4. Exposure Assay. Odor cartridges (2 ml of respective % v/v compound in Paraffin Oil) were placed/replaced in aquariums on days 0, 3.5, 5, and 6. Odor cartridges were made from fresh vortexed aliquots. Plant exposure to volatiles was slowly ramped up in order to compare phenotype changes from low to higher levels of exposure. It was noted that most compounds volatilized out of PO within approximately 24 hours, with residual volatiles remaining within the enclosed chamber.

FIG. 5. First Plant Exposure: Volatile HDAC inhibitor exposure produced varying developmental phenotypes. Root growth and shoot growth data was evaluated over the 7-day exposure period. 2,3-butanedione produced notable lateral root promotion.

FIG. 6. Repeat Plant Exposure: Volatile Odor Compounds with HDAC inhibition demonstrating varying phenotypic changes to Arabidopsis thaliana growth through indirect exposure. Experimental trials demonstrated varying effects on root growth, leaf development, and lateral root promotion.

FIG. 7. VOC displayed phenotypic changes on Arabidopsis thaliana growth through indirect exposure. The minimum sample size after two experiments for each compound was 45 seedlings. Alpha angelica lactone, 2,3-butanedione, and ethyl pyruvate displayed significant root growth inhibition. Alpha angelica lactone, 3,4-hexanedione, ethyl pyruvate, and 2,3-butanedione demonstrate significant phenotypic changes on large leaf growth and lateral root growth. 3.5-day exposure of compounds sufficed to demonstrate significant root growth variation.

FIG. 8. After 7-day exposure all the compounds except 3,4-hexanedione demonstrated significant root growth inhibition.

FIG. 9. Leaf development was counted as the number of large leaves grown in addition to the ones already present from the start of the exposure assay. The trend for the compounds were to inhibit leave development, except for 3,4-hexanedione.

FIG. 10. The number of lateral roots were counted and averages across samples. Alpha angelica lactone, 3,4-hexanedione, and 2,3-butandione promoted lateral root growth. Ethyl pyruvate inhibited both root growth and later root formation.

FIG. 11. A549 Cancer cell line was tested in triplicates at 33 mM and 100 mM 2,3-butanedione treatments. No significant changes in proliferations were seen.

FIG. 12. A549 Cancer cell line was tested in triplicates at 33 mM 2,3-butanedione, ethyl pyruvate, and propyl formate treatments. Significant changes in proliferations were noted with 33 mM 2,3-butanedione treatment this time around.

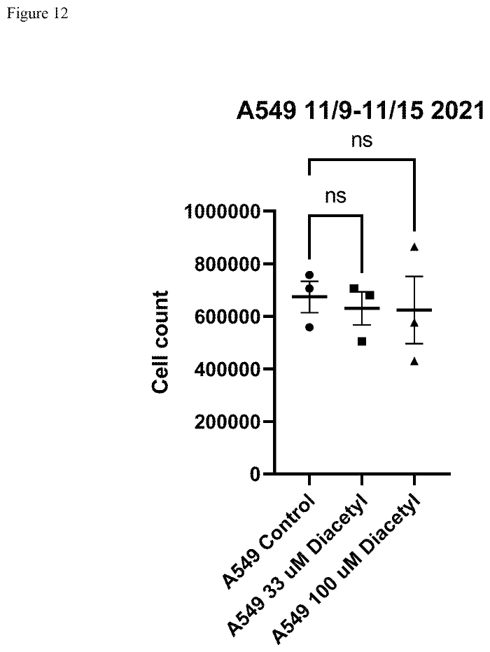

FIG. 13. SK-MEL-5 Cancer cell line was tested in triplicates at 33 uM and 100 uM 2,3-butanedione treatments. No significant changes in proliferations were seen.

FIG. 14. PC3 Cancer cell line was tested in triplicates at 33uM and 100 uM 2,3-butanedione treatments. No significant changes in proliferations were seen.

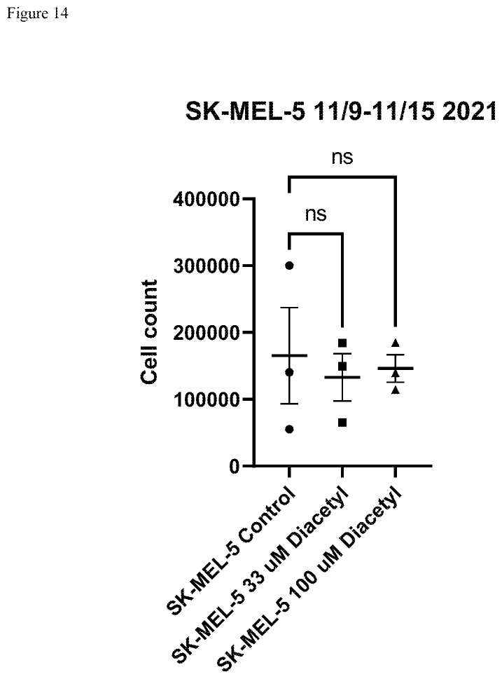

FIG. 15. PC3 Cancer cell line was tested in sextuplicate at 100 uM 2,3-butanedione treatments. Significant changes in proliferations were seen.

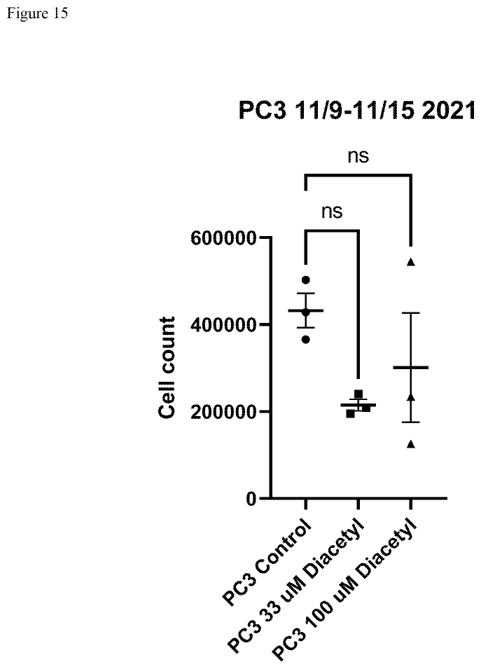

FIG. 16. PC3 Cancer cell line was tested in triplicates at 33 micro-molar 2,3-butanedione, ethyl pyruvate, and propyl formate treatments. No Significant changes in proliferations were noted.

FIG. 17. SH-SY5Y Cancer cell line was tested in triplicates at 33 micro-molar and 100 micro-molar 2,3-butanedione treatments. Significant changes in proliferations were seen in both treatment concentrations.

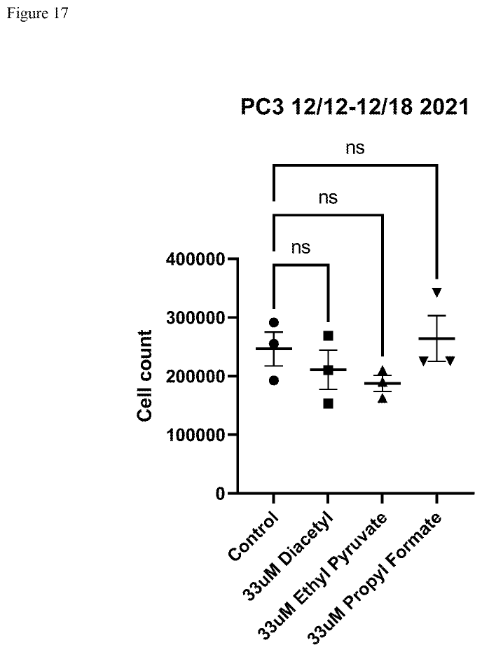

FIG. 18. SH-SY5Y Cancer cell line was tested in sextuplicate at 100 micro-molar 2,3-butanedione treatments. Significant changes in proliferations were seen compared to control.

FIG. 19. SH-SY5Y Cancer cell line was tested in sextuplicate at 33 micro-molar and 100 micro-molar 2,3-butanedione treatments. Significant changes in proliferations were seen compared to control.

FIG. 20. SH-SY5Y Cancer cell line was tested in sextuplicate at 33 micro-molar and 100 micro-molar 2,3-butanedione treatments. Significant changes in proliferations were seen compared to control.

FIG. 21. SH-SY5Y Cancer cell line was tested in triplicates at 33 micro-molar 2,3-butanedione, ethyl pyruvate, and propyl formate treatments. Significant changes in proliferations were noted only for 33 micro molar 2,3-butanedione treatment

FIGS. 22A-22C. Air passed over headspace above (FIG. 22A) 0.1% or (FIG. 22B) 1% solution of diacetyl and into mice cages for 5 days. Brain RNA-seq show changes in expression of a number of genes, in a dose dependent manner. N=2 replicates. (FIG. 22C) GO-enrichment analysis of the DEGs that show increased (UP) or decreased (DOWN) abundance in B.

FIG. 23. Mean fold change of select neuroblastoma related genes in the brain of mice exposed to diacetyl from FIG. 22B.

FIGS. 24A-24C. Show data froM Example 5. (FIG. 24A) Schematic of diacetyl exposure protocol for transcriptome analysis of Arabidopsis leaflets. (FIG. 24B) Plot highlighting up-and down-regulated genes in the diacetyl-exposed groups in leaves, and (FIG. 24C) Bar graphs denoting the protein classification of the plant genes up-and down-regulated after odor exposure.

FIGS. 25A-25C. Roots respond to a volatile diacetyl. (FIG. 25A) Photograph of the odor exposure setup for Arabidopsis thaliana seedlings, and the mean PPM dose of diacetyl based on weight loss measurements. (FIG. 25B) Mean root length and mean number of lateral roots after odorant exposure from the headspace of solutions at indicated concentrations. (FIG. 25C) Transcriptome comparison of Arabidopsis thaliana roots exposed to diacetyl vapors for 7 days from the headspace of the 1% (v/v) dose. Plot highlighting up- and down-regulated genes in the diacetyl-exposed group. Red and blue dots represent up-regulated genes (false discovery rate (FDR)<0.05, logz fold change (LFC)>1) and down-regulated genes (FDR<0.05, LFC<1), respectively. (C) Bar graphs denoting the protein classification of the genes up- and down-regulated after odor exposure.

FIG. 26A-26D. Related odorants differentially inhibit HDACs and alter root development. (FIG. 26A) Structures. (FIG. 26B) In vitro assays with purified human Class I and II HDACs. Mean % inhibition to 15 mM of each volatile compound is shown. N=3, error bars=std. deviation. (FIG. 26C) Representative photographs of the seedlings with colored marks indicating length of primary roots at time points after start of the volatile treatment. (FIG. 26D) Mean root length. E. Counts of lateral root outgrowths. N≥49 One-way analysis of Variance (ANOVA) between groups was performed for plant exposure quantitative data using Graph Pad Prism. 3.5 Day *p-value=0.0239, ****p-value<0.0001

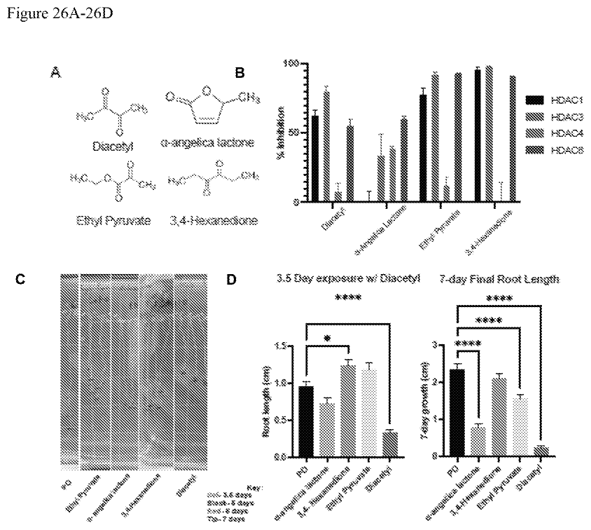

FIGS. 27A-27C. Leaves respond to the HDAC-inhibitory volatile compounds. (FIG. 27A) Schematic of diacetyl exposure protocol for transcriptome analysis of Arabidopsis leaflets. (FIG. 27B) Plot highlighting up- and down-regulated genes in the diacetyl-exposed group. Red and blue dots represent up-regulated genes (false discovery rate (FDR)<0.05, log₂ fold change (LFC)>1) and down-regulated genes (FDR<0.05, LFC<1), respectively. (FIG. 27C) Bar graphs denoting the protein classification of the genes up-and down-regulated after odor exposure.

FIGS. 28A-28C. Volatiles alter flowering times and improve response to freezing. (FIG. 28A) Representative images of Arabidopsis thaliana plants at different stages of beginning to flower, and the mean number of days to start flowering when exposed to vapors from headspace of indicated compounds at 0.1%. One-way analysis of Variance (ANOVA) between groups was performed for plant exposure quantitative data using Graph Pad Prism. N≥6. ****p-value<0.0001. D. Error bars=s.e.m. (FIG. 28B) Representative images of Arabidopsis thaliana plants after cold treatment, and the mean number of days to start flowering when exposed to vapors from headspace of indicated compounds at 0.1%. One-way analysis of Variance (ANOVA) between groups was performed for plant exposure quantitative data using Graph Pad Prism. N≥7. *=p-value=0.0346. (FIG. 28C) Model for volatile odorants affecting HDACs and gene expression in roots and shoots of plants.

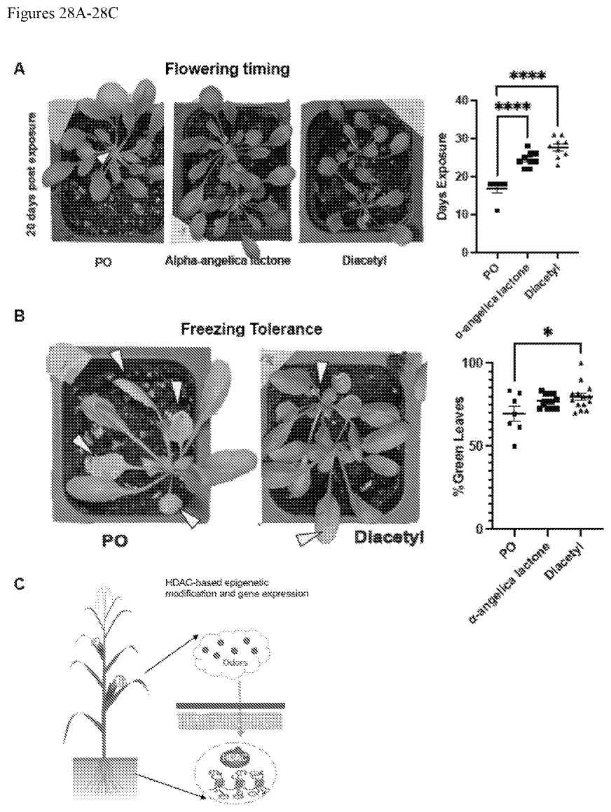

FIG. 29. Volatiles at higher concentrations kill plants. Images of Arabidopsis thaliana plants exposed to headspace vapors for 7 days from a 1% propyl formate (in paraffin oil), 1% diacetyl (in paraffin oil) or control paraffin oil, photographed after recovery for 4 weeks (Example 7).

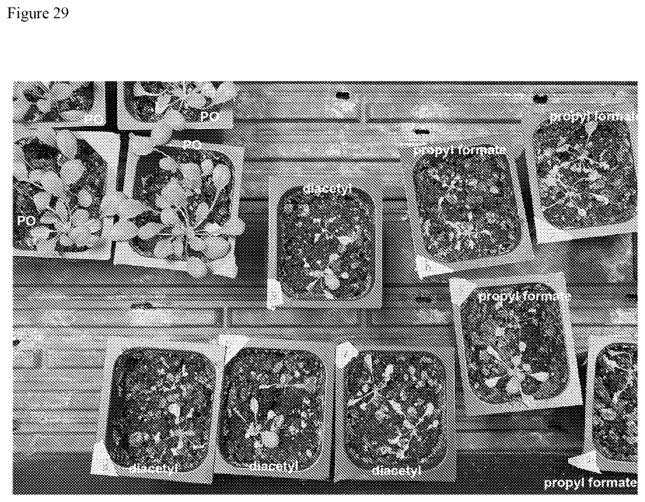

### DETAILED DESCRIPTION

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient In one embodiment, the patient is a human patient.

### Agricultural Applications

Plants can be exposed to compounds that can diffuse as a volatile across short distances (millimeter to meters). The compounds have high vapor pressure and can volatize on their own at around temperatures typically present around live plants. The compounds can be formulated as pellets, capsules, sprays, mixed in water from the irrigation, added to waxes, paints or released from tanks, canisters, and even mounted on unmanned autonomous vehicles that are ariel or on the ground. Additionally, microbes that produce the volatile of interest can be used to administer the compound.

The compounds may be delivered with water or as a neat liquid, solid or solution. The compounds may be sprayed into soil at the time of planting, sprayed into soil at intervals post emergence, or sprayed on the foiliage of a plant. In addition, the compounds may be applied to seeds prior to planting using established methods. For example, they may be coated on seeds with an inert vehicle, tumbled, and dried.

The compounds can be formulated in a variety of ways. For example, they can be formulated as a dustable powder, gel, a wettable powder, a water dispersible granule, a water-dispersable or water-foaming tablet, a briquette, an emulsifiable concentrate, a microemulsifiable concentrate, an oil-in-water emulsion, a water-in-oil emulsion, a dispersion in water, a dispersion in oil, a suspoemulsion, a soluble liquid (with either water or an organic solvent as the carrier), an impregnated polymer film, or other forms known in the art. These formulations may be suitable for direct application or may be suitable for dilution prior to application, said dilution being made either with water, liquid fertilizer, micronutrients, biological organisms, oil or solvent. The compositions are prepared by admixing the active ingredient with adjuvants including diluents, extenders, carriers, and conditioning agents to provide compositions in the form of finely-divided particulate solids, granules, pellets, solutions, dispersions or emulsions. Thus, it is believed that the active ingredient could be used with an adjuvant such as a finely-divided solid, a mineral oil, a liquid of organic origin, water, various surface active agents or any suitable combination of these.

The formulations of the invention can comprise the compounds in any suitable concentration. In one embodiment, for example, the formulation may include the compound in about 95, 90, 80, 60, 50, 40, 30, 20, 10, 5, 2, 1, 0.5 or 0.01 weight percent of the formulation. The formulations may also include other fertilizers or pesticides, which may also be present in any suitable concentration.

The compounds can be applied to soil or plants at any acceptable rate. For example, the compounds can be applied at a rate of at least about 1 kg per acre, at least about 2 kg per acre, at least about 5 kg per acre, at least about 10 kg per acre, or at least about 20 kg per acre, although higher application rates are not excluded.

In one embodiment, the compound can be dissolved in water, organic solvents, or a mixture thereof, without other fertlizers, pesticides, herbicides, or other chemicals in a concentration of from about 2% weight percent of the formulation to about 90% weight percent of the formulation. In another embodiment, the compound can be dissolved in water, organic solvents, or a mixture thereof, without other fertlizers, pesticides, herbicides, or other chemicals in a concentration of from about 20% weight percent of the formulation to about 80% weight percent of the formulation.

In another embodiment, the formulation comprises the compound dissolved in water, organic solvents, or a mixture thereof, with other fertlizers, pesticides, herbicides, or other chemicals present, wherein the compound is present in less than 5% weight percent of the formulation. In another embodiment, the formulation comprises the compound dissolved in water, organic solvents, or a mixture thereof, with other fertlizers, pesticides, herbicides, or other chemicals present, wherein the compound is present in less than 1% weight percent of the formulation.

In certain embodiments, the subject invention further comprises
i) identifying a plant tissue in need of altered development;
ii) examining the plant after administration of the compound to determine if plant tissue development has been altered;
iii) identifying a plant in need or an altered response to an abiotic stress or an infection;
iv) examining the plant after administration of the compound to determine if a response to an abiotic stress or infection has been altered;
v) examining the plant to determine if the administration of the compound has inhibited HDAC; and
vi) measuring the amount of said compound in the air or soil around said plant.

### Target Plants

As used here, the term “plant” includes, but is not limited to, any species of woody, ornamental or decorative, crop or cereal, fruit plant or vegetable plant, flower or tree, macroalga or microalga, phytoplankton and photosynthetic algae (e.g., green algae Chlamydomonas reinhardtii). “Plant” also includes a unicellular plant (e.g., microalga) and a plurality of plant cells that are largely differentiated into a colony (e.g., volvox) or a structure that is present at any stage of a plant's development. Such structures include, but are not limited to, a fruit, a seed, a shoot, a stem, a leaf, a root, a flower petal, etc. Plants can be standing alone, for example, in a garden, or can be one of many plants, for example, as part of an orchard, crop or pasture.

Examples of plants for which the subject invention is useful include, but are not limited to, cereals and grasses (e.g., wheat, barley, rye, oats, rice, maize, sorghum, corn), beets (e.g., sugar or fodder beets); fruit (e.g., grapes, strawberries, raspberries, blackberries, pomaceous fruit, stone fruit, soft fruit, apples, pears, plums, peaches, almonds, cherries or berries); leguminous crops (e.g., beans, lentils, peas or soya); oil crops (e.g., oilseed rape, mustard, poppies, olives, sunflowers, coconut, castor, cocoa or ground nuts); cucurbits (e.g., pumpkins, cucumbers, squash or melons); fiber plants (e.g., cotton, flax, hemp or jute); citrus fruit (e.g., oranges, lemons, grapefruit or tangerines); vegetables (e.g., spinach, lettuce, asparagus, cabbages, carrots, onions, tomatoes, potatoes or bell peppers); Lauraceae (e.g., avocado, Cinnamonium or camphor); and also tobacco, nuts, herbs, spices, medicinal plants, coffee, eggplants, sugarcane, tea, pepper, grapevines, hops, the plantain family, latex plants, cut flowers and ornamentals.

Further types of plants that can benefit from application of the products and methods of the subject invention include, but are not limited to: row crops (e.g., corn, soy, sorghum, peanuts, potatoes, etc.), field crops (e.g., alfalfa, wheat, grains, etc.), tree crops (e.g., walnuts, almonds, pecans, hazelnuts, pistachios, etc.), citrus crops (e.g., orange, lemon, grapefruit, etc.), fruit crops (e.g., apples, pears, strawberries, blueberries, blackberries, etc.), turf crops (e.g., sod), ornamentals crops (e.g., flowers, vines, etc.), vegetables (e.g., tomatoes, carrots, etc.), vine crops (e.g., grapes, etc.), forestry (e.g., pine, spruce, eucalyptus, poplar, etc.), managed pastures (any mix of plants used to support grazing animals).

Further plants that can benefit from the products and methods of the invention include all plants that belong to the superfamily Viridiplantae, in particular monocotyledonous and dicotyledonous plants including fodder or forage legumes, ornamental plants, food crops, trees or shrubs selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g., A. sativa, A. fatua, A. byzantina, A. fatua var. sativa, A. hybrida), Averrhoa carambola, Bambusa sp., Benincasa hispida, Bertholletia excelsea, Beta vulgaris, Brassica spp. (e.g., B. napus, B. rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., E. guineensis, E. oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g., G. max, Soja hispida or Soja max), Gossypium hirsutum, Helianthus spp. (e.g., H. annuus), Hemerocallis fulva, Hibiscus spp., Hordeum spp. (e.g., H. vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g., L. esculentum, L. lycopersicum, L. pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., O. sativa, O. latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g., S. tuberosum, S. integrifolium or S. lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g., T. aestivum, T. durum, T. turgidum, T. hybernum, T. macha, T. sativum, T. monococcum or T. vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

Further examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobromacacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Plants of the embodiments include crop plants (for example, corn, alfalfa, sunflower, Brassica, soybean, cotton, safflower, peanut, sorghum, wheat, millet, tobacco, etc.), such as corn and soybean plants.

Turfgrasses include, but are not limited to: annual bluegrass (Poa annua); annual ryegrass (Lolium multiflorum); Canada bluegrass (Poa compressa); Chewings fescue (Festuca rubra); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerate); perennial ryegrass (Lolium perenne); red fescue (Festuca rubra); redtop (Agrostis alba); rough bluegrass (Poa trivialis); sheep fescue (Festuca ovine); smooth bromegrass (Bromus inermis); tall fescue (Festuca arundinacea); timothy (Phleum pretense); velvet bentgrass (Agrostis canine); weeping alkaligrass (Puccinellia distans); western wheatgrass (Agropyron smithii); Bermuda grass (Cynodon spp.); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

Plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive etc. Leguminous plants include beans and peas. Beans include guar, locust bean, fenugreek, soybean, garden beans, cowpea, mungbean, lima bean, fava bean, lentils, chickpea, etc.

Further plants of interest include Cannabis (e.g., sativa, indica, and ruderalis) and industrial hemp.

All plants and plant parts can be treated in accordance with the invention. Plant parts are understood as meaning all aerial and subterranean parts and organs of the plants such as shoot, leaf, flower and root, examples which may be mentioned being leaves, needles, stalks, stems, flowers, fruit bodies, fruits and seeds, but also roots, tubers and rhizomes. The plant parts also include crop material and vegetative and generative propagation material, for example cuttings, tubers, rhizomes, slips and seeds.

### Weeds

Definition: a wild plant growing where it is not wanted and in competitions with cultivated plants.

Poison sumac (Toxicodendron vernix), Japanese knot weed (Polygonum cuspidatum), crabgrass (Digitaria spp.), dandelions (Taraxaum spp.), broadleaf plantain (Plantago major), common ragweed (Ambrosia artemisiifolia), giant ragweed (Abrosia trifida), bindweed (Convolvulus arvensis), ground ivy (Glechoma hederacea), purslane (Portulaca olearacea), stinging nettle (Urtica dioica), curly dock (Rumex crispus), bitter dock (Rumex obtusifolius) wild madder (Galium mollugo), clover leaf (Trifolium), orange jewelweed (Impatiens capensis), bittersweet (Celastrus spp.), horsetail weed (Equisetum arvense), chickweed (Stellaria media), Canada thistle (Cirsium arvense), annual sow thistle (Sonchus oleraceus), quackgrass (Elymus repens), Shepherd's Purse (Capsella bursa-pastoris), woodsorrel (Oxalis stricta), common mallow (Malva neglecta), lambsquarters (Chenopodium album), pigweed (Amaranthus retroflexus), nutsedge (Cyperus spp.), dayflower (Commelina spp.), velvetleaf (Abutilon theophrasti), wild violet (Viola papilionacea), smartweed (Polygonum pensylvanicum), quickweed (Galinsoga parviflora), pokeweed (Phytolacca americana), black nightshade (Solanum nigrum), black medic (Medicago lupulina), poison ivy (Rhus radicans), burdock (Arctium spp.), common groundsel (Senecio vulgaris), curly dock (Rumex crispus), spurge (Euphorbia maculata), puncturevine (Tribulus terrestris), rough cinquefoil (Potentilla norvegica), sandbur (Cenchrus spp.), sheep sorrel (Rumex acetosella), Bermuda grass (Cynodon dactylon), common daisy (Bellis perennis L.), common self-heal (Prunella vulgaris L.), creeping buttercup (Ranunculus repens), doveweed (Murdannia nudiflora), flatweed (Hypochaeris radicata), ground elder (Aegopodium podagraria), herb bennet (Geum urbanum), marestail (Conyza canadensis).

### Therapeutic Applications

The pharmaceutical compositions of the invention can comprise one or more excipients. When used in combination with the pharmaceutical compositions of the invention the term “excipients” refers generally to an additional ingredient that is combined with the compound to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

The active compounds can be formulated as pharmaceutical compositions and administered to a mammalian host, such as a human patient in a variety of forms adapted to the chosen route of administration, i.e., orally or parenterally, by inhalation or by intravenous, intramuscular, topical or subcutaneous routes.

Thus, the active compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compounds may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

In one embodiment, the compound is administered by inhalation, for example, from a spray, by breaking open a capsule, using a nebulizer, a candle, or an aroma dispenser, or by inhaling from an impregnated mat, sticker, canister, or evaporator, etc.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

In some embodiments, a disease, disorder, or condition treated according to the subject invention may be a peripheral nervous system (PNS) disease or disorder, or a central nervous system (CNS) disease or disorder.

In some embodiments, the compounds according to the present invention may be used to treat a peripheral neuropathy. In some embodiments, the compounds according to the present invention may be used to treat a hereditary peripheral neuropathy such as a Charcot Marie Tooth Disease (CMT). In some embodiments, the compounds according to the present invention may be used to treat an acquired neuropathy such as chemically induced peripheral neuropathy (CIPN). In some embodiments, the compounds according to the present invention may be used to treat diabetic peripheral neuropathy (DPN).

Charcot Marie Tooth Disease (CMT) is a general name for a group of distinct hereditary neuropathies, each of which is associated with one or more diverse mutations specific to the particular form of the disease. The specific types of CMT are grouped into either axonal or demyelinating forms. Specific types of CMT include CMT1, CMT2, CMT3 and CMT4, among others. In turn, these specific types are divided into further subtypes. For example, for CMT2 which is an axonal form, subtypes include CMT2A, CMT2B, CMT2C, CMT2D, CMT2E, CMT2F, CMT2G. CMT2A is the most prevalent of the CMT2 diseases and is associated with mutation of the MFN2 gene located on chromosome 1 which codes for the protein mitofusin 2 involved in mitochondrial fusion. Multiple HDAC6 inhibitors have shown efficacy in transgenic mouse models of CMT including CMT1 and CMT2. See, e.g., Benoy V. et al., HDAC6 is a Therapeutic Target in Mutant GARS-induced Charcot-Marie-Tooth Disease, Brain, 141, 673-687 (2018); d'Ydewalle, C. et al., HDAC6 Inhibitors Reverse Axonal Loss in a Mouse Model of Mutant HSPB1-induced Charcot-Marie-Tooth Disease, Nature Medicine, 17:8, 968-975 (2011); Picci, C. et al., HDAC6 Inhibition Promotes α-tubulin Acetylation and Ameliorates CMT2A Peripheral Neuropathy in Mice, Experimental Neurology, 328, 113281 (2020); Ha, N. et al., A Novel Histone Deacetylase 6 Inhibitor Improves Myelination of Schwann Cells in a Model of Charcot-Marie-Tooth Disease Type 1A, Br. J. Pharmacol., 177 (22), 5096-5113 (2020). In some embodiments, a specific type of CMT can be treated by compounds according to the present invention. In some embodiments, the CMT that can be treated by compounds of the present invention is CMT1. In some embodiments, the CMT that can be treated by compounds of the present invention is CMT2. In some embodiments, the CMT that can be treated by compounds of the present invention is CMT2A.

Chemotherapy-induced peripheral neuropathy (CIPN) is a debilitating neurologic condition caused by a substantial number of cytotoxic chemotherapy drugs. These drugs cause different pathologic insults to neurons, resulting in various conditions including paresthesia, numbness, hypersensitivity, and pain. CIPN manifests itself in about 40% of patients undergoing chemotherapy, and 80% of such patient population experiences persistent CIPN. Furthermore, CIPN is a major limiting factor on chemotherapy tolerability and efficacious dosing. Currently, the standard of care is limited to palliative analgesics, and no disease-modifying therapies exist for CIPN.

The symptoms of CIPN depend on the types of chemotherapy and nerve fibers that are affected. For example, if chemotherapies affect mainly the sensory nerve fibers, symptoms include unusual sensations (paresthesias), numbness, balance problems or pain. In cases where the motor nerves are affected, symptoms may include weakness of the muscles in the feet and hands.

Diagnosis of CIPN is according to methods known in the art, e.g., based on patient history, clinical examination, and/or laboratory tests. These include, but are not limited to, electromyography with nerve conduction studies, skin biopsies to evaluate cutaneous nerve innervation, and nerve and muscle biopsies for histopathological evaluation.

In some embodiments of the method of treating CIPN according to the present invention, an effective amount of a compound according to the present invention or a composition comprising same is administered in a substantially simultaneous manner with a chemotherapy drug. The chemotherapy drugs associated with CIPN include, but are not limited to, paclitaxel, eribulin, bortezomib, cis-platin, and/or vincristine. In other embodiments, a chemical entity according to the present invention is administered to a subject after a chemotherapy drug has been administered to the subject.

In some embodiments, the compounds according to the present invention may be used to treat a neuromuscular disease. In some embodiments, the neuromuscular disease is a hereditary muscular neuromuscular disease. In some embodiments, the neuromuscular disease is Duchenne Muscular Dystrophy (DMD). In some embodiments, the neuromuscular disease is Becker Muscular Dystrophy (BMD).

In some embodiments, the present invention provides a method for treating a neurodegenerative disorder in a subject, comprising administering to the subject an effective amount of a compound according to the present invention or a composition comprising same.

In some embodiments, a neurodegenerative disorder is a peripheral neuropathy. In some embodiments, the peripheral neuropathy is CIPN, CMT, or DPN.

In some embodiments the neurodegenerative disease is a CNS disease. In some embodiments the CNS disease is ALS.

In another aspect, the present invention provides a method for treating an inflammatory disease, disorder, or condition in a subject, comprising administering to the subject an effective amount of a compound according to the present invention or a composition comprising same.

In some embodiments, inflammatory diseases, disorders, or conditions include rheumatoid arthritis.

In another aspect, the present invention provides a method for treating cancer in a subject, comprising administering to the subject an effective amount of a compound according to the present invention or a composition comprising same.

In some embodiments, the cancer is a hematological cancer. In other embodiments the cancer is a solid tumor cancer. In some embodiments the cancer is a breast cancer or an ovarian cancer.

In another aspect, the present invention provides a method for treating pain in a subject, comprising administering to the subject an effective amount of a compound according to the present invention or a composition comprising same.

In some embodiments, pain arises from a variety of sources including neuropathic pain (such as post herpetic neuralgia, nerve injury/damage, the “dynias”, e.g., vulvodynia, phantom limb pain, root avulsions, painful diabetic neuropathy, compressive mononeuropathy, ischemic neuropathy, painful traumatic mononeuropathy, or painful polyneuropathy), central pain syndromes (potentially caused by virtually any lesion at any level of the nervous system), postsurgical pain syndromes (e.g., postmastectomy syndrome, postthoracotomy syndrome, stump pain), bone and joint pain (osteoarthritis, rheumatoid arthritis, ankylosing spondylitis), repetitive motion pain, carpal tunnel syndrome, dental pain, cancer pain, myofascial pain (muscular injury, fibromyalgia), perioperative pain (general surgery, gynecological), chronic pain, dysmenorrhea, as well as pain associated with angina, and inflammatory pain of varied origins (e.g., osteoarthritis, rheumatoid arthritis, rheumatic disease, teno-synovitis and gout), headache, and migraine.

In some embodiments, the present invention provides a combination therapy to treat a disease, disorder, or condition in a subject. The term “combination therapy” refers to the administration of two or more therapeutic agents to a subject in need of treatment of a disease, disorder, or condition described herein, where the therapeutic agents comprise at least one compound of the present invention. In some embodiments, two or more therapeutic agents may treat the same disease, disorder, or condition. In other embodiments, two or more therapeutic agents may treat more than one disease, disorder, or condition.

Administration in a combination therapy encompasses co-administration of the two or more therapeutic agents in a substantially simultaneous manner, such as in a single formulation having a fixed ratio of the two or more therapeutic agents or in separate formulations for each of the two or more therapeutic agents. In addition, such administration also encompasses use of each type of therapeutic agent in a sequential or separate manner, either at approximately the same time or at different times.

In some embodiments of the present invention involving combination therapies, the mechanisms of action of the pharmaceutical agents having a therapeutic action may be the same or different. In some embodiments of the present invention, the combination comprises at least one of the compounds according to the present invention and at least another therapeutic agent that is not an inhibitor of HDAC. In other embodiments of the present invention, the combination comprises at least one of the compounds according to the present invention, and at least a different therapeutic agent that is an inhibitor of HDAC and not a compound according to the present invention. In exemplary embodiments, where a disease, disorder, or condition being treated is cancer, the combination comprises a chemotherapy drug and at least one of the compounds according to the present invention. In further embodiments, the combination comprising an established cancer drug therapy and at least one of the compounds according to the present invention provides synergistic effects of treating cancer.

As used herein, the term “subject” includes an animal, preferably a mammal, and more preferably a human. In some embodiments, humans include prenatal human forms. In some embodiments, a subject is suffering from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is a fetus, an infant, a child, a teenager, an adult, or a senior citizen (i.e., the subject is of advanced age, such as older than 50). In some embodiments, a child refers to a human between two and 18 years of age. In some embodiments, an adult refers to a human eighteen years of age or older.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

An “inhibitory amount” is meant an amount of compound sufficient to exert an inhibitory effect as measured by, for example, an assay such as the ones described herein.

The activity of a compound utilized in a method according to the present invention may be assayed in vitro or in vivo. An in vivo assessment of the efficacy of the compounds of the invention may be made using an animal model of a disease or disorder, e.g., in a mouse or rat rodent model. Cell-based assays may be performed using, e.g., a cell line isolated from a tissue that expresses HDAC, or a cell line that recombinantly expresses HDAC. Additionally, biochemical or mechanism-based assays, e.g., measuring cAMP or cGMP levels, Northern blot, RT-PCR, etc., may be performed. In vitro assays include assays that determine cell morphology, protein expression, and/or the cytotoxicity, enzyme inhibitory activity, and/or the subsequent functional consequences of treatment of cells with compounds of the invention. Alternate in vitro assays quantify the ability of the inhibitor to bind to protein or nucleic acid molecules within the cell. Inhibitor binding may be measured by radiolabelling the inhibitor prior to binding, isolating the inhibitor/target molecule complex and determining the amount of radiolabel bound. Alternatively, inhibitor binding may be determined by running a competition experiment where new inhibitors are incubated with purified proteins or nucleic acids bound to known radioligands. The aforementioned assays are exemplary and not intended to limit the scope of the invention. A person skilled in the art can appreciate that modifications can be made to conventional assays to develop equivalent assays that obtain the same result.

Those skilled in the art appreciate that identification and/or characterization of desirable or effective compounds commonly involves assessment of one or more activities in an animal model. Commonly employed models relevant to work described herein include, for example, animal models of CIPN, DPN, CMT, and Duchenne Muscular Dystrophy.

The particular dose and dosage regimen designed to deliver an effective amount of a compound according to the present invention are appropriately decided, taking into account factors such as age, sex, and particulars of the subject, as well as the condition, the disorder, the disease, and the disease state involved, and whether the purpose is preventative. As used herein, the terms “effective amount,” and “effective dose” are used to refer to an amount of something (e.g., a compound, a composition, time) that is capable of causing a desired outcome (e.g., reduce symptoms of the disease, disorder, or condition in an individual).

Administration of an effective amount of the compound according to the present invention may be accomplished in daily or multi-daily doses of the chemical entity over a period of a few days to months, or even years. If the compound is administered with another therapeutic agent, the effective amount of the compound may carry the same range as is typical for use of that compound as a monotherapy, or the amount may be lower than a typical monotherapy amount especially if the combination therapy results in a synergy.

Unless otherwise specified, the word “includes” (or any variation thereon, e.g., “include”, “including”, etc.) is intended to be open-ended. For example, “A includes 1, 2 and 3” means that A includes but is not limited to 1, 2 and 3.

Unless otherwise specified, the phrase “such as” is intended to be open-ended. For example, “A can be a halogen, such as chlorine or bromine” means that A can be, but is not limited to, chlorine or bromine.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention, e.g., the ability to improve the bioavailability of a substance. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example, within 2 standard deviations of the mean. As further examples, “about” can be understood as within 10%, 9%, 8%, 7%, 6%, 5%, 4%, 3%, 2%, 1%, 0.5%, 0.1%, 0.05%, or 0.01% of the stated value. The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The invention will now be illustrated by the following non-limiting Examples.

### EXAMPLES

### Example 1

### In-Vitro HDAC Inhibitor Screening

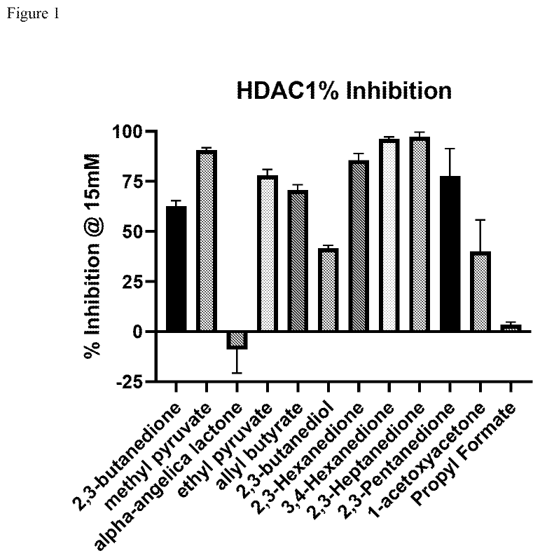

Presented are data for selected compounds that act as Histone deacetylase inhibitors in vitro or HDAC inhibitors. 26 compounds were screened against purified human HDACs. Each displayed varying forms of HDAC inhibition against Class I and Class II HDACs at 15 mM concentration in 1% DMSO (FIGS. 1-4). Most of the tested compounds demonstrated HDAC inhibition, to differing extents to each of the 4 purified HDACs tested.

### HDAC inhibition Methods

HDAC activity of class I HDACs (HDAC1 and 3) was measured with the fluorometric HDAC Activity Assay kit: HDAC1 (10011563, Cayman Chemical, Ann Arbor, MI) and HDAC3 (50073 BPS Bioscience, San Diego, CA), according to the manufacturer's instructions. HDAC activity of class II HDACs (HDAC4 and HDAC6) was measured with the fluorometric HDAC Activity Assay kit: HDAC4, HDAC 4 (50064 and 50076, BPS Bioscience, San Diego, CA), according to the manufacturer's instructions. All compounds were tested in single dose duplicate mode at 15 mM concentrations in 1% DMSO, based on the IC₅₀ of 2,3-butanedione established in earlier work. Due to interference, concentrations of >30 mM were not used in the IC₅₀ calculation. To account for any interference at lower concentrations, all baseline measurements of blank wells (no HDAC enzyme) were performed and subtracted with the various concentrations of 2,3-butanedione.

### Example 2

### HDAC Inhibitors Affect Plant Gene Transcription and Growth Results

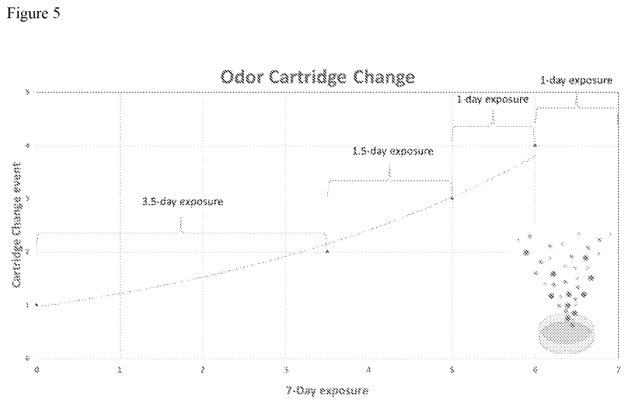

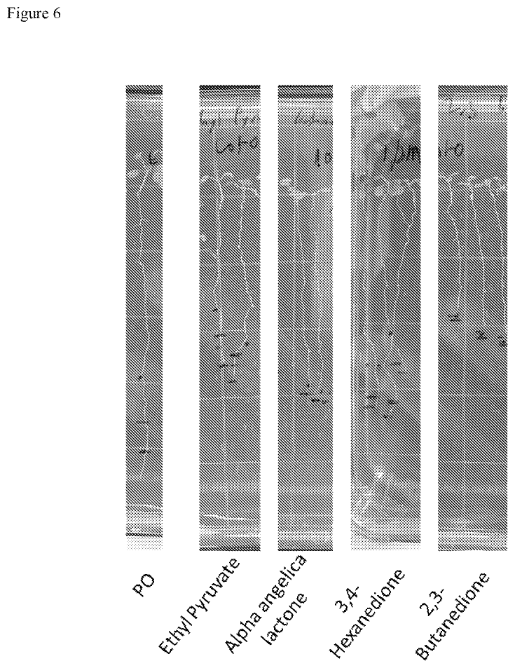

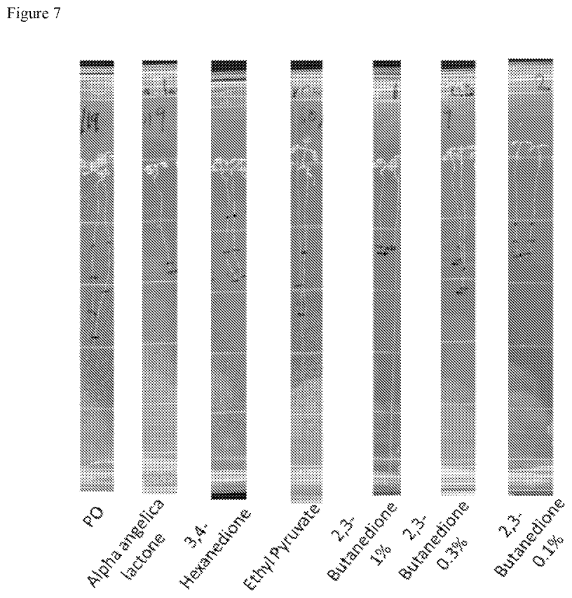

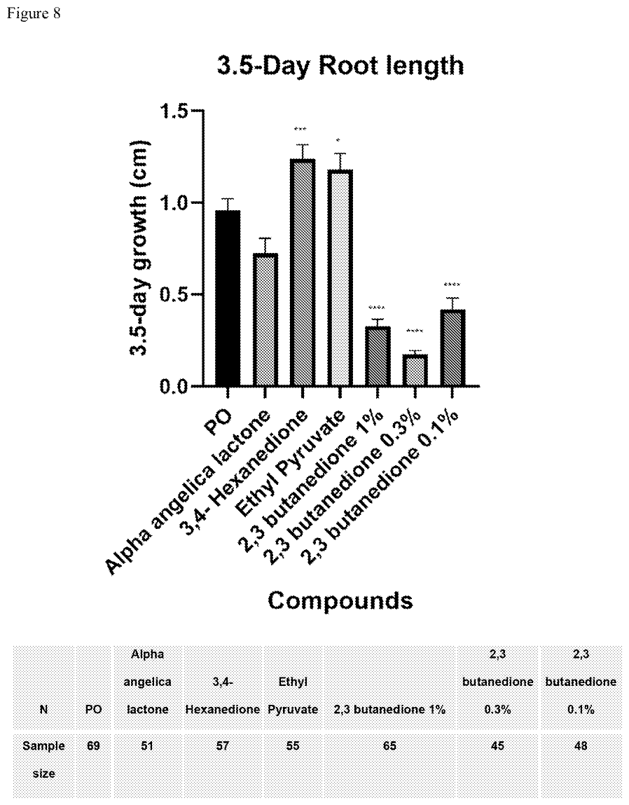

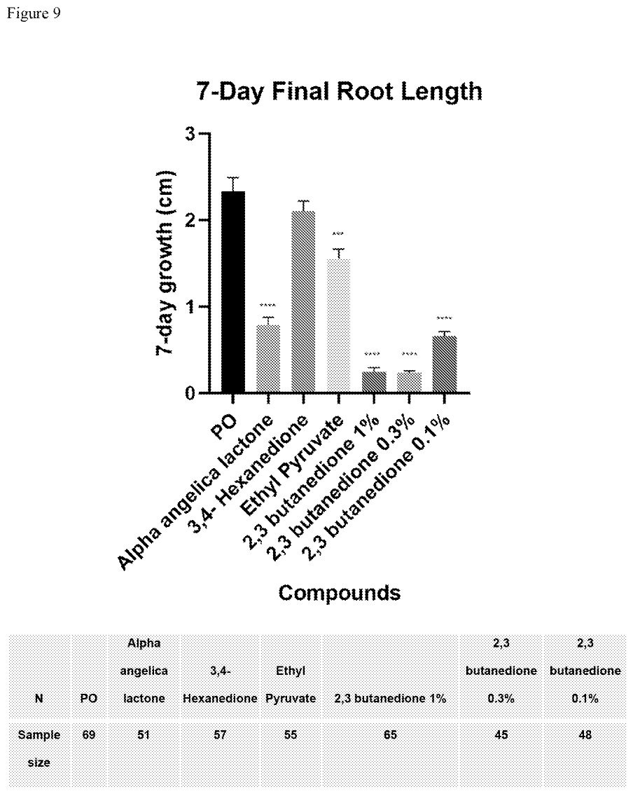

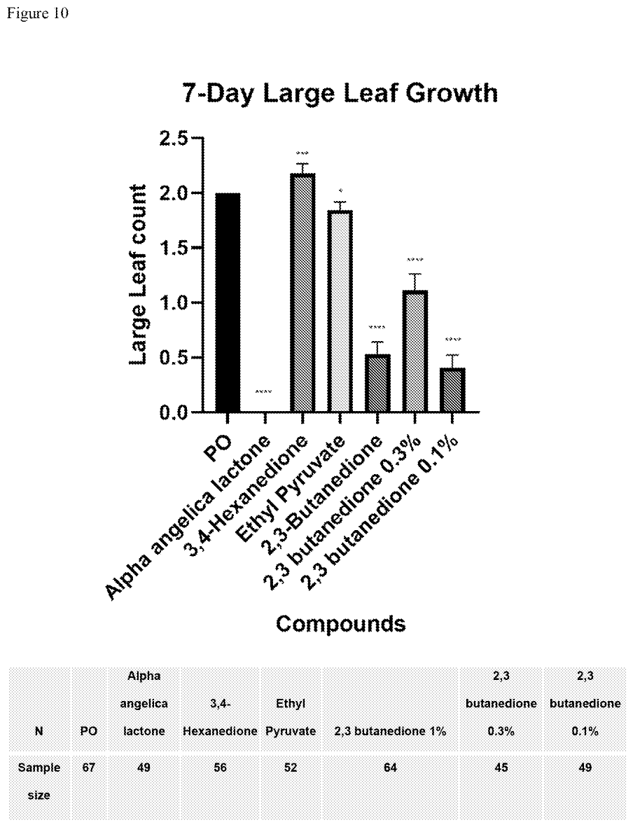

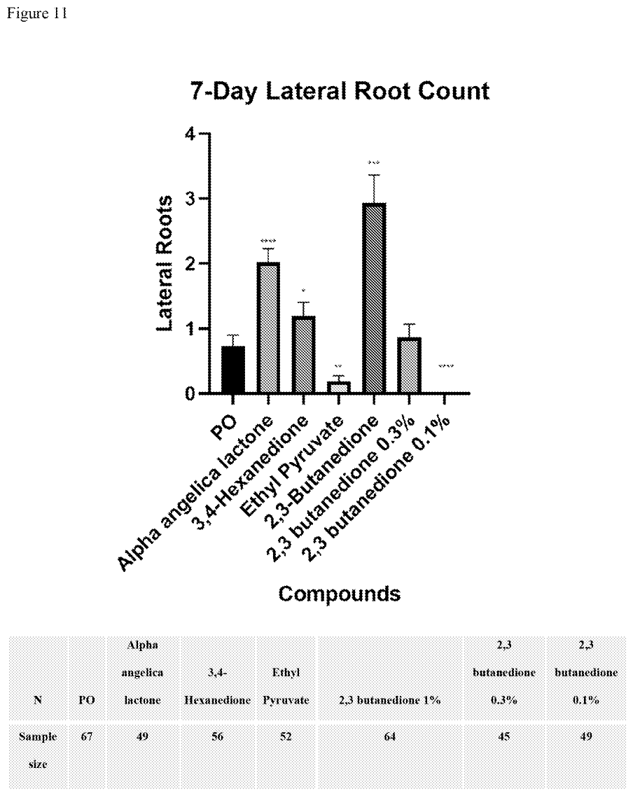

Four volatile HDAC inhibitors (2,3-butanedione, ethyl pyruvate, alpha angelica lactone, and 3,4 hexanedione) were tested against Arabidopsis thaliana seedlings. These compounds were selected due to the variations in compound structures and HDAC inhibition profiles. A range of phenotypes was found on plant development over a 7-day volatile exposure period using the compounds, diluted around 1% volume/volume in Paraffin oil odor cartridges. Volatile exposure alone sufficed to display significant changes in root and leaf development (FIGS. 5-11). This represents a novel, indirect delivery method of using volatile compounds to alter gene expression in eukaryotes.

### Method: Arabidopsis thaliana Experimental Exposure

The Odor Exposure assay consists of controlled exposure of volatile odor compounds to model plant Arabidopsis within enclosed aquariums and similar growth conditions (23° C. with a 12-hour light/dark cycle). For Arabidopsis, seedlings were aligned on agar plates for growth after a 4-day cold treatment process. The agar plates with seedlings were then placed in enclosed aquariums with moist paper towels soaked in 10 ml of DI water for humidity. The plates were placed along the wall, approximately 12 cm from odor dispensers which resided in the center of the glass tank.

The test compound odor dispensers, consisting of (60×15 mm) polystyrene Petri dishes containing 2 ml of 1% v/v in PO, are replaced at the 3.5, 5, and 6-day time mark during the 7-day experiment. Root growth is marked during each time mark and the plates were imaged. The plants are to be collected after the 7-day exposure, photographed, and the leaves/roots carefully dissected separated, flash frozen in liquid nitrogen, and preserved for future sequencing. Root growth is measured using ImageJ.

### Results: HDAC Inhibitory Volatiles Alter Shoot and Root Development in Plants

To test whether plants also show observable phenotypes following exposure to HDAC inhibitory volatiles, Arabidopsis seedlings were used as a model to evaluate changes in development of shoots and roots. The RNA-seq analysis from leaflets of Arabidopsis seedlings revealed significant changes in gene expression in response to 2,3-butanedione exposure, affecting a variety of genes that could confer changes in the developmental program. Since shoot and root growth phenotypes are easy to score, the effects of 2,3-butanedione and three additional HDAC inhibitory odorants identified in vitro, each with different functional groups: α-angelica lactone, ethyl pyruvate, and 3,4-hexanedione were tested. The seedlings were exposed to the headspace of the test odorants (1%) and paraffin oil (PO) solvent, within enclosed aquariums and similar growth conditions. Root tips were marked during 4 time points and plates photographed. The plants were collected after the 7-day exposure, photographed, and the leaves/roots were separated. Exposure to all 4 HDAC inhibitory odorants tested led to phenotypic differences when compared to control PO exposed. Interestingly, ethyl pyruvate and 2,3-butanedione showed opposite effects on lateral roots showing inhibition and increase, respectively. α-Angelica lactone, 3,4-hexanedione, and 2,3-butanedione also showed changes in the number of true leaf development/growth. These volatile HDAC inhibitors showed varying HDAC inhibition levels in vitro for HDAC 1, 3, 4, and 6, which may contribute to the observed phenotypic differences.

Additionally, a dose response study was carried out with 2,3-butanedione headspaces from 0.1%, 0.3%, and 1% solutions. Even at a 10-fold lower dose, there was a significant change from the solvent treated seedlings in every growth category. Taken together, these experiments demonstrate that volatiles of these HDAC inhibitors produced distinct phenotypic changes in Arabidopsis thaliana seedlings, presumably due to altered gene expression.

### Example 3

### Cancer Cell Treatment

Broad spectrum HDAC inhibitors were first FDA approved for cancer treatment, with many new publications supporting their efficacy against inhibiting a variety of cancers. The ability of HDAC inhibiting compounds to inhibit cell proliferation was assessed using the cell lines shown below. Data suggests that 2,3-butanedione has potential in inhibiting neuroblastoma (SH-SY5Y) and prostate cancer (PC3) cell lines.

| | | Media Type |
|---|---|---|
| Cell line | | (all with Final |
| Names | Cancer type | volume of 10% FBS) |
| A549 | adenocarcinoma human | F12K |
| | alveolar basal epithelial | |
| | cells | |
| SH-SY5Y | neuroblastoma cell line | DMEM:F12 |
| | from a metastatic bone | |
| | tumor | |
| PC3 | bone metastasis of grade | RPMI |
| | IV of prostate cancer | |
| SK-MEL-5 | melanoma cell line | EMEM |

### Cancer Cell Viability Assay Protocol

Cells were maintained in a 37° C. incubator with 5% CO₂ throughout the experiment. Compound stocks were dissolved in 10% DMSO, but final concentrations were made fresh in complete media as needed. 10% DMSO solvent was used as control in complete media. Cells were seeded onto 12-well plates and allowed 12-24 hours until treatment began to allow cells to adhere to wells. This was followed by minimum 5-day treatment of compounds, changing the media with treatment every 48 hours. Cell lines A549, PC3, and SK-MEL-5 were seeded at 8000 cells/well which would provide 80-100% confluency after 6-day growth. SH-SY5Y was seeded at 8000 cells/well then 16,000 cells/well due to slower growth and given at least 10 days for sufficient growth. After allotted growth times, cell counts were performed via Countess™ automated cell counter. All assays were completed with a minimum of triplicate replicates for all conditions and analyzed through GraphPad Prism One-way ANOVA for significance.

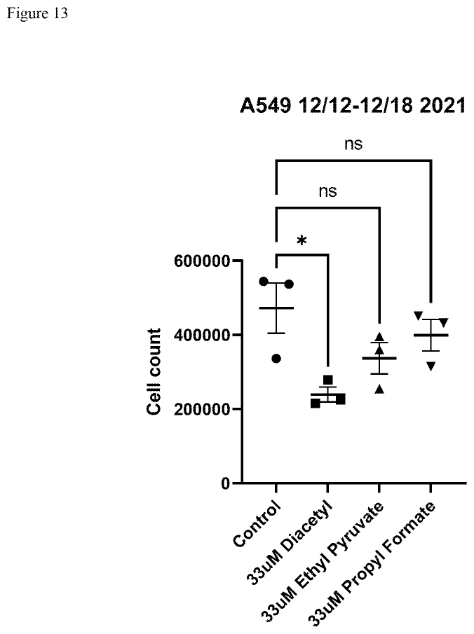

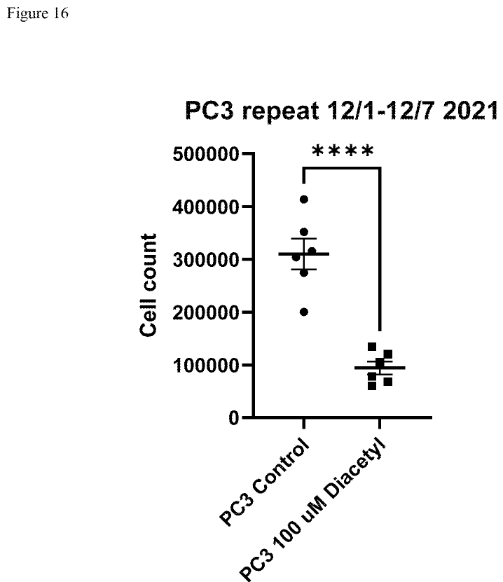

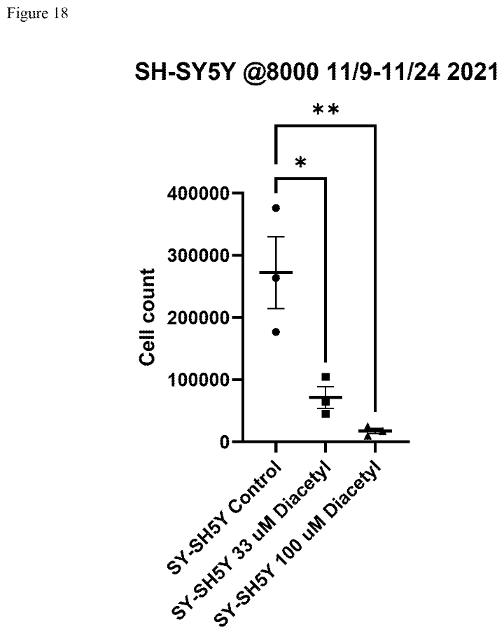

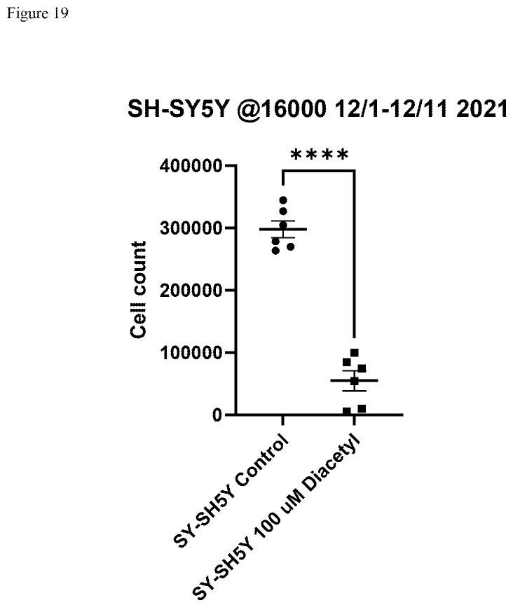

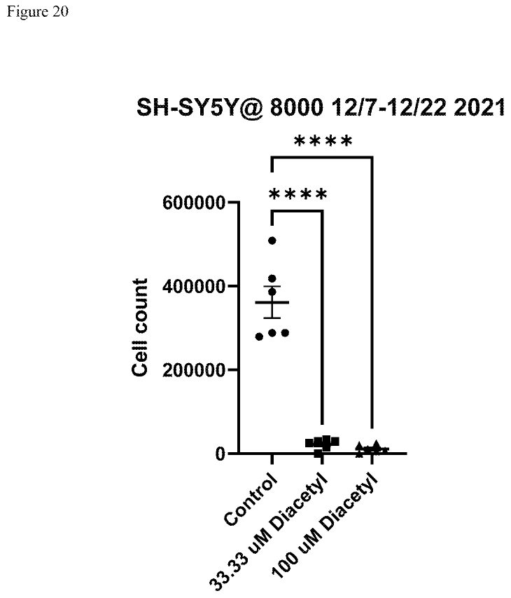

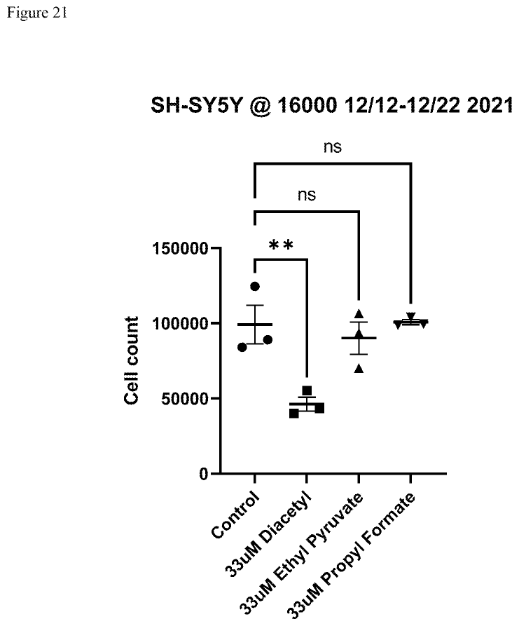

Data suggest significant inhibition of cell growth was seen in 2,3-butanedione treatments in SH-SY5Y cancer cell lines at 33 micro-molar and 100 micro-molar concentrations (FIG. 18-21). 2,3-butanedione treatments in PC3 cancer cell lines showed significant inhibition at 100 micro-molar concentration treatments (FIG. 16). There may be some A549 inhibition with 2,3-butanedione treatment at 33 micro-molar (FIG. 13).

### Example 4

### Gene Expression Changes by 2,3-Butanedione Exposure Reduce Expression of Important Oncogenes Which Can be Used to Treat Variety of NMyc Dependent Cancers

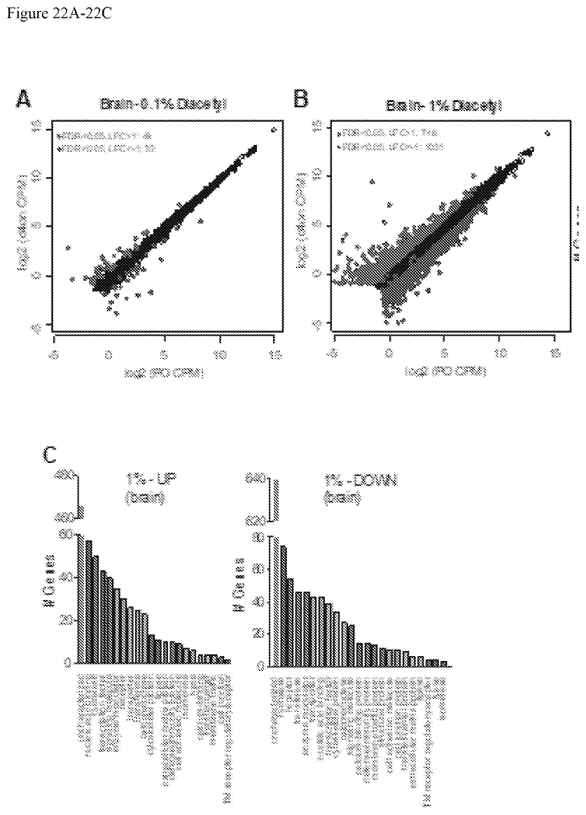

HDAC inhibitors are an important class of drugs being tested for a number of different conditions including neurodegenerative diseases and brain cancers. A major design challenge for such drugs is the ability to cross the blood-brain barrier. An interesting possibility that arises due to the volatility and small size of odorants is that they could diffuse through the intranasal route to the brain directly. To test whether cells in the brain respond to diacetyl vapors by altering gene expression RNA-seq experiments were carried out on mice exposed only to aroma of diacetyl for 5 days. Littermate controls were exposed in a similar manner to the solvent (PO) headspace. Several genes were differentially expressed upon exposure to 0.1% diacetyl (49 up-regulated, 32 down regulated, |log₂ fold-change|>1, FDR<0.05) or to 1% diacetyl (748 up-regulated, 1031 down regulated, |log₂ fold-change|>1, FDR<0.01) (FIG. 22A). GO analysis of the regulated genes revealed several interesting sets of genes were significantly altered in each set (FIG. 22B).

### Key Genes Associated with Neuroblastomas are Downregulated in the Diacetyl Exposed Mouse Brain Tissue

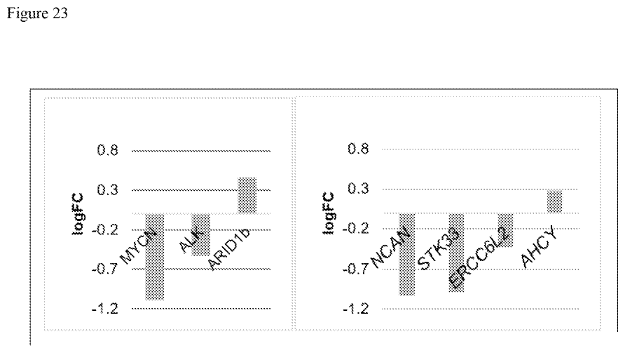

Several genetic and “omics” studies have identified genes that are upregulated in neuroblastomas, some of which are considered to key. The DEGs from the diacetyl-exposed mouse brain transcriptome for several of these key genes were evaluated. Half of the six key genes identified across multiple studies were significantly altered, two of which were significantly downregulated, MycN (P=2.06E-13, FDR=1.76E-12) and Alk (P=3.3E-5,FDR=9.48E-5) (FIG. 23). An unbiased computational study evaluated omics datasets and identified four genes that were the best predictors of clinical outcome, all of which are significantly different in the brains of the diacetyl exposed mice, strikingly 3 of which were significantly downregulated, NCAN (p=1.33E-16, FDR=1.59E-15), STK33 (p=4.6E-7,FDR=1.79E-6) and ERCC6L2 (p=4.57E-5, FDR=0.00012) (FIG. 23). These findings are extremely provocative, given that the downregulated genes, particularly MYCN play a role in neuroblastomas and in several other cancers.

### Example 5

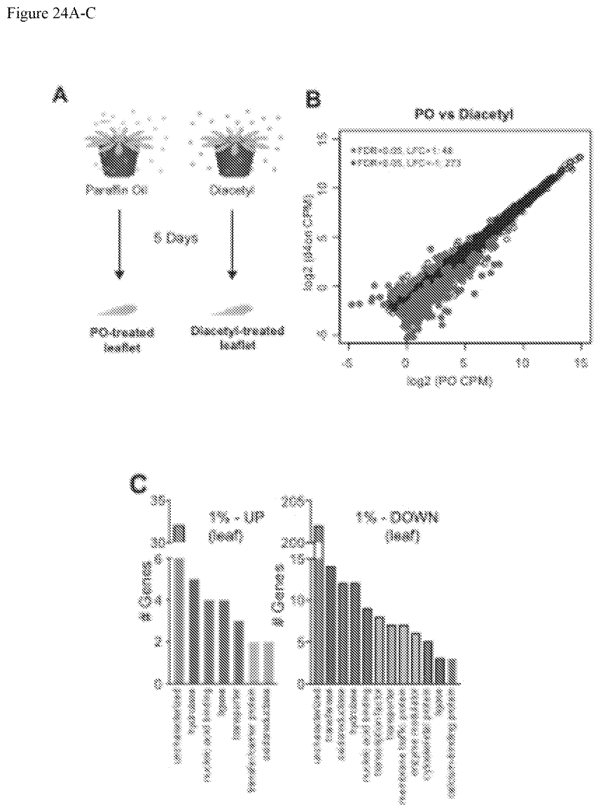

A. thaliana plants (Col-0) that had been transplanted at day 7 to soil were placed in the experimental room for 2 days to acclimate (23°° C. with a 12-hour light/dark cycle). A single potted plant was placed in a 4-liter clear glass jar, with openings attached to a vacuum to allow for air exchange. A small beaker containing 1 ml of 1% diacetyl in PO or PO alone were placed inside the jar. Plants were watered at 2.5 days. After 5 days, individual leaflets were dissected, and flash frozen in liquid nitrogen. Trizol-based RNA isolation was carried out as above. Two biological replicates were performed for each condition, with an average of 68,355,820 reads/replicate, with an average of 95% mapped. Multiplexed libraries were made from total RNA input using the Illumina TruSeq RNA sample preparation kit (v2) and 50 bps single-end sequencing was done using the NextSeq500. Data is shown in FIGS. 24A-C.

### Example 6

In accordance with the subject invention, some volatiles identified here can be detected by plants and cause changes in gene expression. The volatiles act on the epigenome by acting as inhibitors of Histone Deacetylases, presumably altering chromatin structure. These epigenetic changes affect the development and morphology of the roots, leaving a long-term effect, the shoots also respond to volatiles with changes in gene expression and alterations in leaf and flower development. In one embodiment, volatiles alleviate leaf damage caused by freezing. The ability to reprogram epigenetics in plants using volatiles that can be easily delivered provides a highly advantageous tool to improve agriculture in a scalable manner.

### Exposure to Diacetyl Vapors Alters Root Development and Gene Expression

In order to study the physiological effects of long-term exposure to microbial volatiles the model system Arabidopsis was exposed for 5 days to diacetyl, a volatile chemical emitted by fermentation by yeast or microbial degradation in the soil associated with roots (R. J. Morcillo et al., EMBO J 39, e102602 (2020); M. A. Farag, et al., Phytochemistry 67, 2262-2268 (2006); and C. M. Ryu et al., P Natl Acad Sci USA 100, 4927-4932 (2003). The Odor Exposure assay consisted of controlled exposure of volatile HDAC inhibiting compounds to model plant Arabidopsis within enclosed aquariums and similar growth conditions. The test compound odor cartridges, consisting of 60×15 mm polystyrene Petri dishes containing 2 ml of indicated concentration in Paraffin Oil, were replaced at the 3.5, 5, and 6-day time mark during the 7-day experiment (FIG. 1A, left). In separate experiments the weight loss from the odor cartridges over time was measured and used it to calculate the concentration of diacetyl in the glass chambers in parts per million (FIG. 1A, right). These levels are comparable to sources where measurements have been taken previously.

Root growth was marked during each point and the plates were photographed. The plants were collected after the 7-day exposure, photographed, and the roots were separated. Visible phenotypic differences were seen in the root length and numbers of lateral roots in a dose dependent manner comparison to the control paraffin oil exposed plants (FIG. 1B). In the presence of diacetyl vapors, the primary root length shows a lack of growth with the higher dose showing the most significant loss. Interestingly, the lateral roots show the opposite effect and at the highest dose are nearly 3 times more numerous (FIG. 1B).

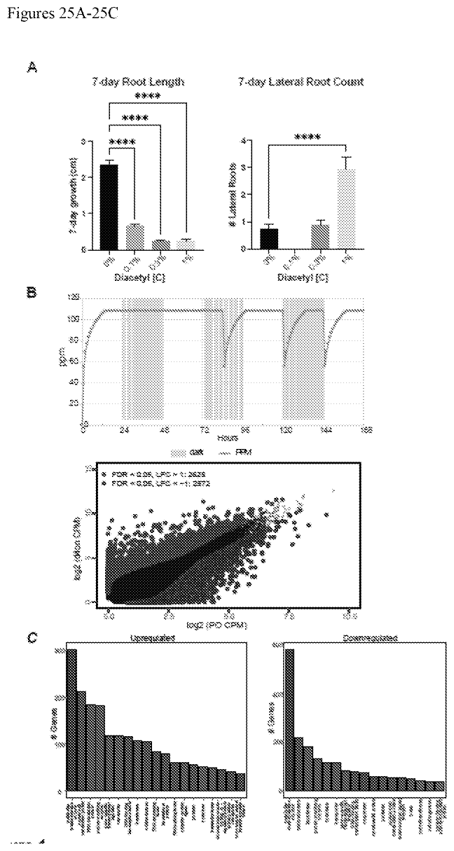

In order to understand the molecular changes occurring with the volatile exposure and possibly underlying this developmental phenotype, an RNA-Seq analysis of the roots in diacetyl exposed vs control was performed. A large number of differentially expressed genes (DEGs) was found in the roots of the diacetyl vapor exposed Arabidopsis. 5197 differentially expressed genes (DEGs) (false discovery rate, FDR<0.05) were identified in the root transcriptome of diacetyl-exposed seedlings compared to control. Of these, 2625 genes were significantly up-regulated (log₂ fold-change>1; red dots in FIG. 25C) and 2572 genes were significantly down-regulated (log₂ fold-change<−1; blue dots in FIG. 25C). A broad range of genes was significantly altered, with several biological process GO terms significantly enriched in the up-regulated gene list including “metabolic interconversion enzymes”, “nucleic acid metabolism” and “RNA metabolism”. In the down-regulated gene set the GO term most enriched was “metabolic interconversion enzymes” and “oxidoreductases”.

### VOCs Inhibit HDAC Family Members Differentially and Affect Root Development

A 2-D structural comparison of diacetyl to known bioactive compounds revealed that it is similar to a β-hydroxybutyrate, which is a known inhibitor of histone deacetylase enzymes (HDACs) and is produced by the liver (FIG. 25E) (T. Shimazu et al., Science 339, 211-214 (2013)). HDAC inhibitors are known to modulate gene expression broadly by promoting the acetylation of lysine residues on histone tails, thereby promoting accessible chromatin structure. In mammals, β-hydroxybutyrate has been shown to induce changes in the expression of several genes.

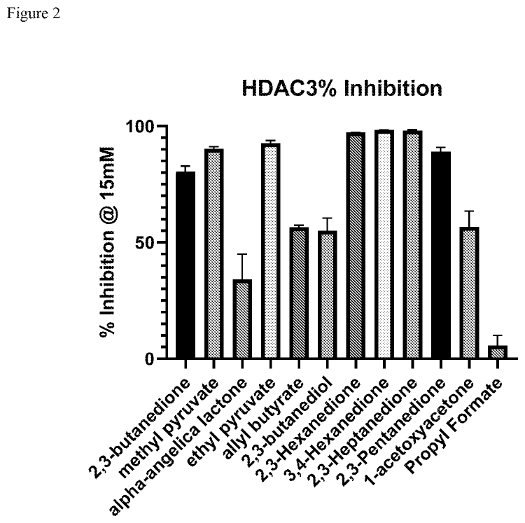

Diacetyl and 3 structurally related VOCs were evaluated using in vitro assay kits for measuring activity of four purified HDACs. Of the four VOCs), each has a different functional group, two are natural in origin, previously detected from microbes that are known to be present in soil (M. C. Lemfack et al., Nucleic Acids Res 46, D1261-D1265 (2018); and R. H. Cagan, W. N. Zeiger, Proc. Nat. Acad. Sci. U.S.A. 75, 4679-4683 (1978)) and two that are not (α-angelica lactone and ethyl pyruvate). Ethyl pyruvate, while structurally related, is actually a potent antimicrobial acting on bacteria and fungi (T. Debebe et al., PLOS One 11, e0162919 (2016)). All 4 VOCs inhibited both Class I HDACs (L. L. Walling, The Myriad Plant Responses to Herbivores. J Plant Growth Regul 19, 195-216 (2000); and M. Heil, R. Karban, Trends Ecol Evol 25, 137-144 (2010)) and the Class II HDAC6 with differences in degree (FIG. 2A,B). The α-angelica lactone inhibited Class II HDACs more strongly than Class I. These findings suggest that these HDAC-inhibitory VOCs could potentially effect gene expression in the roots and therefore affect their development. These findings fit well with a past study using non-volatile soluble drugs that showed HDACs function in primary root elongation and root emergence by modulating auxin-mediated development (H. N. Nguyen, et al., Plant Cell Rep 32, 1625-1636 (2013)).

In order to directly evaluate whether exposure to vapors of the four HDAC-inhibitory VOCs affect root development, we performed a set of experiments using A. thaliana seedlings as before. Solutions for each of the four chemicals on a small petri dish were placed away from the seedlings growing in agar plate and enclosed in glass tank. The Root tips were marked at specified days post treatment start and photographs analyzed. The plants were collected after the 7-day exposure, photographed, and the roots were separated. Detailed measurements were made from the photos using ImageJ and the root length and numbers of lateral roots quantified at day 3.5 and day 7 after start if the treatment (FIG. 1B). In the presence of diacetyl vapors, the primary root length shows a lack of growth with the higher dose showing the most significant loss. Interestingly, the lateral roots show the opposite effect and at the highest dose are nearly 3 times more numerous (FIG. 1B).

### Exposure to VOCs Alters Shoot Development and Gene Expression

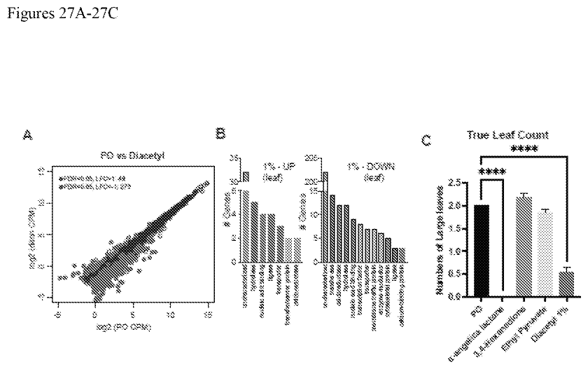

The HDACs are also important for gene expression in the shoots as well. Young plants in pots were exposed to vapors of diacetyl from a solution of the same concentration as before and performed an RNASeq analyses. 321 genes were differentially regulated in the leaflet of Arabidopsis thaliana following exposure to volatile diacetyl for 5 days (FDR<0.05). These DEGs are distributed across multiple chromosome locations and represent genes of diverse molecular functions (FIG. 27C). Taken together, these results indicate that HDACs serve as highly conserved pathways to transduce detection of VOCs like diacetyl, resulting in specific alteration of gene expression in both roots and shoots.

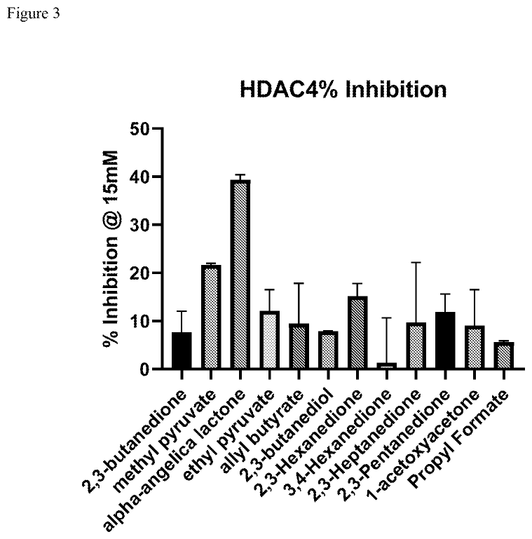

Next the number of true leaves in the seedling VOC exposure assay were counted as before, after 7 days of VOC exposure. A variety of phenotypes were seen, with a-angelica lactone and diacetyl showing significant decreases relative to the control paraffin oil (solvent PO) exposed plants (FIG. 3C). Interestingly 3,4-hexanedione showed a significant increase in the number of leaves, while ethyl pyruvate seemed to have no effect (FIG. 3C). These results show that the development of leaves in the shoot are also affected by the HDAC inhibitory volatiles.

### Effect of Identified VOCs on Adult Plants: Flowering and Cold Resistance

Dramatic effects of VOCs have been seen in the seedling stage. The effects on with flowering timing were observed, since the process involves numerous changes in gene expression and therefore potentially susceptible to HDAC inhibitory volatiles. Mutants in HDACs like HDA5 show delayed flowering due to upregulation of flowering repressor genes like FLC and MAF1 (M. Luo et al., Plant J 82, 925-936 (2015)). Conversely, mutants in HDA9lead to early flowering due to increased expression of FLOWERING LOCUS T (FT) and AGAMOUS-LIKE 19 (AGL19) (M. J. Kang, et al., New Phytol 206, 281-294 (2015)). We placed 3 weeks old A. thaliana plants in the glass chambers and exposed them to the vapors of two of the VOCs, diacetyl and α-angelica lactone volatilizing from 0.1% solutions in the glass chamber.

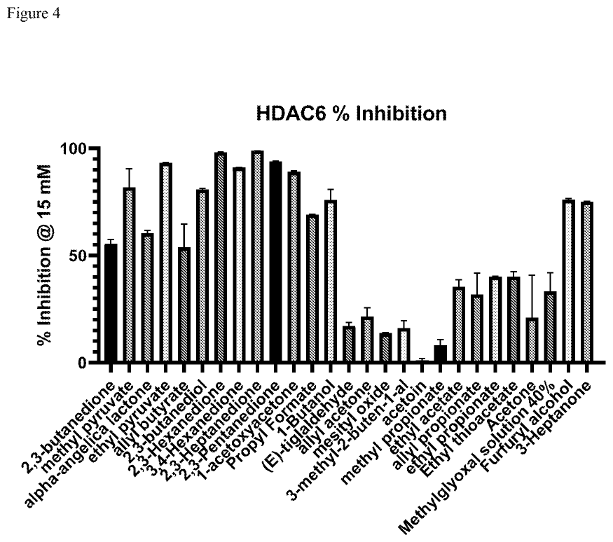

The plants in the treatments showed a significant and substantial delay in the number of days it took to flower. Exposure to volatiles from a-angelica lactone, which inhibits only Class 2 HDACs strongly, showed approximately a week delay in flowering. Plants exposed to diacetyl, which inhibits both class 1 and class 2 HDACs, showed a 11-day delay in flowering (FIG. 4A,B). These results indicate that these volatiles can show significant effects on flowering even at minute doses. While the levels were too low to calculate using the weight loss method, we can expect it to be as low as ˜10 ppm in the glass chambers (FIG. 1B).

HDACs have also been implicated in responses to a number of abiotic stresses, such as temperature, drought, salt, etc in a variety of ways (M. Luo, et al., Frontiers in Plant Science 8, (2017)). One of the phenotypes that can be rapidly tested in a laboratory is tolerance to cold temperatures in Arabidopsis. HD2C has been shown to lower expression of COR genes under warm temperatures by hypoacetylation of their promoters, and is degraded under cold stress leading to hyperacetylation of H3 in COR and expression (J. Park et al., Proceedings of the National Academy of Sciences 115, (2018)). In order to test whether HDAC-inhibitory volatiles of the subject invention can affect cold-tolerance, we set up an experiment where we cold-acclimatized the plants for 7 days at 4° C., and then transferred them to respective odor exposure chambers in a freezing chamber and gradually dropped to −10° C., and then covered with ice and placed in the dark for 24 hours at 4° C. and then transferred under the light at 23° C. as done before (J. Park et al., Proceedings of the National Academy of Sciences 115, (2018)). The yellowing of leaves was determined after 5 days and the controls ˜30% of the leaves turned yellow-brown in the classic cold response previously documented. In the case of the exposure to diacetyl vapors, the plants showed a significantly lower number of yellow leaves (˜20%) suggesting that the HDAC inhibitory volatiles had some degree of protection (FIG. 4C,D).

According to the subject invention, VOCs can directly enter cells and alter the epigenetic landscape to bring about changes in gene expression. The VOCs act as inhibitors of histone deacetylases (HDACs), a class of enzymes that plays a crucial role in regulating chromatin.

In histone tails, lysine acetylation (ac) and methylation, are amongst the two key post translational modifications that play key roles in regulating gene expression, and involve a complex programming (S. L. Berger, Current Opinion in Genetics & Development 12, 142-148 (2002)) of temporal sequences and combinatorial spatial patterns that regulate chromatin state (T. Kouzarides, Current Biology 13, R895-R898 (2003)), such as open, closed, poised (S. C. Elgin, S. I. Grewal, Heterochromatin: silence is golden. Current Biology 13, R895-R898 (2003)). Such epigenetic changes significantly influence gene expression, enabling transcriptional plasticity, in response to environmental exposure to certain odorants. Epigenetic regulation also leads to a certain programmed chromatin state that is transmitted through cell division thus exerting a longer-term influence (T. A. Rando, H. Y. Chang, Cell 148, 46-57 (2012); and V. Calvanese, et al., Ageing Research Reviews 8, 268-276 (2009)).

### Example 7: Effect of Identified Volatiles on Adult Plants: Weed Killing Assay

Since large scale changes in gene expression occur it is presumed that higher doses of some of the compounds could negatively affect some plants, even causing death. Since Arabidopsis thaliana is also considered a type of weed and goes by the common name Thale cress, it was used as a model to test the weed killing potential at higher doses. In order to evaluate the activity of some of these volatiles in plant killing twenty-seven day old A. thaliana plants (Col-0), that had been transplanted at day 7 to soil, were placed in 10-gallon glass aquarium as chamber along with a petridish containing 2 ml of chemical dissolved in PO at 1% (volume/volume) or PO alone were placed at the other side of the container. Plants were exposed to odor vapors for 7 days and observed for death. The exposed potted were observed for 4 weeks afterwards for regrowth and photographed. At the end of the recovery period propyl formate was determined to be the most effective at killing (7 of 8 plants dead) as observed by dead leaves. Diacetyl was effective at killing 6 of 8 of the exposed plants, while the age matched controls plant showed no death (FIG. 25).

### MATERIALS AND METHODS

### Arabidopsis thaliana Experimental Exposure

The Odor Exposure assay consists of controlled exposure of volatile odor compounds to model plant Arabidopsis within enclosed aquariums and similar growth conditions (23° C. with a 12-hour light/dark cycle). The test compound odor cartridges, consisting of (60×15 mm) polystyrene Petri dishes containing 2 ml of 1% v/v in paraffin oil (PO), were replaced at the 3.5, 5, and 6-day time mark during the 7-day experiment. Root growth was marked during each time point and the plates were imaged. Numbers of true leaves were also counted. The plants are to be collected after the 7-day exposure, photographed, and the leaves/roots carefully dissected separated, flash frozen in liquid nitrogen, and preserved for future sequencing. Root growth is measured using ImageJ.

### Odor Exposure for RNA-seq

A. thaliana plants (Col-0) that had been transplanted at day 7 to soil were placed in the experimental room for 2 days to acclimate (23° C. with a 12-hour light/dark cycle). A single potted plant was placed in a 4-liter clear glass jar, with openings attached to a vacuum to allow for air exchange. A small beaker containing 1 ml of 1% diacetyl in PO or PO alone were placed inside the jar. Plants were watered at 2.5 days. After 5 days, individual leaflets were dissected, and flash frozen in liquid nitrogen.

A. thaliana plants (Col-0) were grown in an Agar medium were synchronized to germinate using pre-treatment in cold. Exposure experiments were started using plants the plates when they were 11 days old and placed in a 10-gallon glass aquarium as a chamber along with a petri dish containing 1 ml of chemical dissolved in PO or PO alone were placed at the other side of the container. After the desired exposure, the roots were separated, and a Trizol-based RNA isolation was carried out as above. Two biological replicates were performed for each condition, with an average of 68,355,820 reads/replicate, with an average of 95% mapped. Multiplexed libraries were made from total RNA input using the Illumina TruSeq RNA sample preparation kit (v2) and 50 bps single-end sequencing was done using the NextSeq500.

### Flowering Assay

Twenty-seven day old A. thaliana plants (Col-0), that had been transplanted at day 7 to soil, were placed in 10-gallon glass aquarium as chamber along with a petridish containing 2 ml of chemical dissolved in PO or PO alone were placed at the other side of the container. Plants were exposed to odors until all plants began to flower, with odor cartridges being replaced daily. Signs of flower buds opening were counted as plant flowering. One-way analysis of Variance (ANOVA) between groups was performed for plant exposure quantitative data using Graph Pad Prism 9.

### Cold Tolerance Assay

Twenty six day old A. thaliana plants (Col-0), that had been transplanted at day 7 to soil, were cold-acclimatized the plants for 7 days at 4° C., and then transferred to respective odor exposure chambers with a petridish containing 2 ml of chemical dissolved in PO or PO alone in a freezing chamber in which temperatures went down every 2° C. for 0.5 h and maintained for 1 hour, and it dropped up to −10° C. Afterwards, the plants were covered with ice and placed in the dark for 24 hours at 4° C. and then transferred under the light at 23° C. The survival rate was determined after 5 days. The percentage of the yellow-brown leaves to the total number of leaves were compared in test vs control (solvent) exposed. One-way analysis of Variance (ANOVA) between groups was performed for plant exposure quantitative data using Graph Pad Prism 9.

### Weed Killing Assay

Twenty-seven day old A. thaliana plants (Col-0), that had been transplanted at day 7 to soil, were placed in 10-gallon glass aquarium as chamber along with a petridish containing 2 ml of chemical dissolved in PO at 1% (volume/volume) or PO alone were placed at the other side of the container. Plants were exposed to odor vapors for 7 days and observed for death. Pots were observed for 4 weeks afterwards for regrowth and photographed.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method of: 1) altering development of plant tissue in a plant, 2) altering response to abiotic stress, or infection, in a plant, or 3) killing a plant, wherein said method comprises contacting the plant with an effective amount of a volatile HDAC inhibitor selected from alpha-angelica lactone, 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, or methylglyoxal, benzyl nicotinate, a flavone, phenethyl resorcinol, acacetin, eriodictyol, phloretin, 2-phenylethyl 2-aminobenzoate, aromadedrin, benzoyl peroxide, heliotropyl phenyl acetate, chalcone, benzyl para-anisate, cinnamyl anthranilate, (Z)-2-penten-1-yl benzoate, 2-naphthyl phenyl ketone, bergamottin, isoimperatorin, para-cresyl benzoate, guaiacyl benzoate, caffeic acid ethyl ester, 4-phenyl propyl pyridine, 2-phenylethyl benzoate, benzyl benzoate, 2-methyl butyl salicylate, phenethyl sorbate, para-anisaldehyde, methyl anthranilate schiff's base, bibenzyl ether, ortho-anisyl benzoate, cinnamyl benzoate, 1-(2-hydroxy-4-methoxyphenyl)-3-(pyridin-2-yl) propan-1-one, ethyl 4-methylphenoxyacetate, benzyl trans-2-methyl-2-butenoate, alpha,alpha-dimethyl anisyl acetone, styryl acetate, amyl cinnamate, ortho-cresyl benzoate, methyl phenyl methylene aminobenzoate, phenethyl pyridine, (Z)-N-coumaroyl-5-hydroxyanthranilic acid, homoeriodictyol, benzyl 2,3-dimethyl-2-butenoate, meta-cresyl benzoate, (Z)-anisylidene acetone, guaiacyl phenyl acetate, 3-phenyl propyl salicylate, geranyl cantabiline, cortex pyridine, ethyl para-cresyl carbonate, ethyl phenoxyacetate, hesperetin, 3′,7-dihydroxy-4′-methoxyflavan, allyl (Z)-cinnamate, bisdemethoxycurcumin, amyl salicylate, phenyl acetaldehyde, methyl anthranilate schiff's base, p-tolyl phenylacetate, 4-formyl phenyl butyrate, 4-methoxybenzyl phenylacetate, para-cresyl 3-oxo-3-phenyl propionate, (E)-2-hexen-1-yl salicylate, furfuryl phenyl acetate, butyl 2-naphthyl ether, (E)-cinnamyl acetate, cinnamyl acetate, ethyl (E)-cinnamate, ethyl cinnamate, 2-para-cresyl oxyethyl acetate, neryl anthranilate, cinnamaldehyde, methyl anthranilate schiff's base, cinnamyl tiglate, (E)-cinnamyl propionate, 1-(4-Methoxyphenyl)-1-penten-3-one, imperatorin, para-cresyl valerate, meta-cresyl phenyl acetate, 3-methyl-3-buten-1-yl benzoate, para-anisyl acetoacetate, isoamyl p-methoxycinnamate, isoeugenyl benzoate, (Z)-3-hexen-1-yl cinnamate, eugenyl benzoate, isoamyl salicylate, phenylmethyl benzeneacetate, curcumin, butyl cinnamate, cinnamyl acetoacetate, para-cresyl phenyl ether, citral, methyl anthranilate schiff's base, cinnamyl valerate, xanthohumol, allyl phenoxyacetate, 2-phenylethyl 3-phenyl-2-propenoate, geranyl benzoate, (E)-3-butylidene phthalide, geranyl cinnamate, neryl cinnamate, cinnamyl cinnamate, amyl anthranilate, (E)-2-hexen-1-yl formate, (E)-3-hexen-1-yl formate, (E)-9-tetradecen-1-yl formate, (E)-cinnamyl formate, (E,Z)-2,6-nonadien-1-yl formate, (Z)-3-hexen-1-yl formate, (Z)-9-tetradecen-1-yl formate, 1,3,4,5,6,7-hexahydro-1,1,5,5-tetramethyl-2H-2,4a-methanonaphthalen-7-yl formate, 1-hexyl allyl formate, 2,6-dimethylhept-5-enyl formate, 2-decalinyl formate, 2-melozol formate, 2-methoxyethyl formate, 2-methyl butyl formate, 2-naphthyl formate, 2-octyl formate, 2-pentyl formate, 2-phenoxyethyl formate, 3-cyclohexyl 3-hexenyl formate, 3-mercapto-3-methyl butyl formate, 3-octyl formate, 3-phenyl propyl formate, 4-tert-amyl cyclohexyl formate, 4-tert-butyl cyclohexyl formate, allyl formate, alpha-amyl cinnamyl formate, alpha-campholenyl formate, aluminum triformate, amber formate, ammonium formate, amyl formate, benzyl formate, bornyl formate, butyl chloroformate, butyl formate, calcium formate, carvyl formate, caryophyllene formate, cedryl formate, chitosan formate, cholesteryl formate, cinnamyl formate, citronellyl formate, cuminyl formate, cyclodecyl formate, cyclododecyl formate, cyclohexyl formate, cyclooctyl formate, cymbopogon martini herb extract formate, decahydro-1,1,7-trimethyl-3a, 7-methano-3ah-cyclopentacyclooct-3-yl formate, decyl formate, dextro-bornyl formate, dihydrocarvyl formate, dihydrocitronellyl formate, dihydromyrcenyl formate, dimethyl benzyl carbinyl formate, dodecyl formate, ethyl 4-methyl-5-thiazolyl formate, ethyl benzoyl formate, ethyl chloroformate, ethyl formate, ethyl orthotrithioformate, ethyl vanillyl formate, eugenyl formate, fenchyl formate, furfuryl formate, geranyl formate, heliotropyl formate, heptyl formate, herbal cyclohexane formate, hexahydromethanoindenyl formate, hexahydrotetramethyl methanonaphthalene-8-methyl formate, hexyl formate, isoamyl formate, isobornyl formate, isobutyl formate, isodecyl formate, isoeugenyl formate, isononyl formate, isopropyl formate, isopulegyl formate, laevo-menthyl formate, linalyl formate, longifolene formate, marine formate, melozol formate, menthadienyl formate, menthyl formate, methyl formate, myristyl formate, myrtenyl formate, neryl formate, nonan-3-yl formate, nonisyl formate, nonyl formate, nopyl formate, octyl formate, oleyl formate, (±)-3-methyl-gamma-decalactone, (±)-chrysanthemolactone, (±)-dihydromint lactone, (±)-isoalantolactone, (±)-pantolactone, (±)-trans-whiskey lactone, (+/−)-mint lactone, (−-)-cis-whiskey lactone, (−)-isomint lactone, (−)-mint lactone, (1R)-chrysanthemolactone, (1S)-chrysanthemolactone, (E)-dairy lactone, (R)-(−)-pantolactone, (R)-delta-decalactone, (R)-delta-dodecalactone, (R)-delta-hexalactone, (R)-delta-nonalactone, (R)-delta-octalactone, (R)-delta-undecalactone, (R)-epsilon-decalactone, (R)-gamma-decalactone, (R)-gamma-dodecalactone, (R)-gamma-heptalactone, (R)-gamma-hexalactone, (R)-gamma-nonalactone, (R)-gamma-octalactone, (R)-gamma-undecalactone, (R)-gamma-valerolactone, (R)-massoia lactone, (S)-delta-decalactone, (S)-delta-dodecalactone, (S)-delta-hexalactone, (S)-delta-nonalactone, (S)-delta-octalactone, (S)-delta-undecalactone, (S)-epsilon-decalactone, (S)-gamma-decalactone, (S)-gamma-dodecalactone, (S)-gamma-heptalactone, (S)-gamma-hexalactone, (S)-gamma-nonalactone, (S)-gamma-octalactone, (S)-gamma-undecalactone, (S)-gamma-valerolactone, (S)-massoia lactone, (Z)-dairy lactone, (Z)-gamma-jasmolactone, (Z)-yuzu lactone, (Z,E)-nepetalactone, 1beta-hydroxyalantolactone, 2-acetyl butyrolactone, 2-C-methyl-1,4-erythrono-D-lactone, 2-decen-1,4-lactone, 2-hydroxyalantolactone, 2-isobutyl-gamma-valerolactone, 23-hydroxyphysalolactone, 3-isobutenyl-5-methyl-5-vinyl butyrolactone, 3-methyl butyrolactone, 4-ethyl-gamma-octalactone, 4-hydroxy-3-(methylthio) butanoic acid lactone, 5-hydroxy-7-decenoic acid lactone, 9-hydroxy-gamma-tetradecalactone, alantolactone, alpha-decalactone, alpha-methyl-gamma-butyrolactone, animal carbolactone, beta-angelica lactone, beta-methyl-delta-valerolactone, beta-propiolactone, blumealactone A, blumealactone B, blumealactone C, butyrolactonethiol, cassialactone, catalpalactone, cis,cis-nepetalactone, cis-oak lactone, costus lactone, costus valerolactone, creamy lactone, curcumalactone, D-mevalonolactone. dairy lactone, dehydrocostus lactone, dehydrodiferulic dilactone, dehydroindicolactone, delta-2-dodecenolactone, delta-decalactone, delta-dodecalactone, delta-gluconolactone, delta-heptalactone, delta-hexalactone, delta-juniper lactone, delta-nonalactone, delta-octadecalactone, delta-octalactone, or delta-tetradecalactone or a mixture thereof.

2. The method of claim 1, which further comprises one or more of:
i) identifying a plant tissue in need of altered development;
ii) examining the plant after administration of the compound to determine if plant tissue development has been altered;
iii) identifying a plant in need or an altered response to an abiotic stress or an infection;
iv) examining the plant after administration of the compound to determine if a response to an abiotic stress or infection has been altered;
v) examining the plant to determine if the administration of the compound has inhibited HDAC; and
vi) measuring the amount of said compound in the air or soil around said plant.

3. The method of claim 1, wherein the volatile HDAC inhibitor has a vapor pressure >0.001 mm Hg at 25 C.

4. The method of claim 1, wherein the method alters development of plant tissue, or alters response to abiotic stress, or infection, in the plant, and wherein the plant is contacted with an effective amount of alpha-angelica lactone, 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, diacetyl, or methylglyoxal, or a mixture thereof.

5. The method of claim 1, wherein the tissue is primary root, secondary root, leaves, fruits, flowers or seeds.

6. The method of claim 1, wherein the abiotic stress is cold, heat, flooding, drought, or wind.

7. The method of claim 1, wherein the infection is fungi, bacteria or virus.

8. The method of claim 1, which is a method of killing a plant, wherein the method comprises first identifying a plant to be killed and then contacting the plant with 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, methylglyoxal, benzyl nicotinate, a flavone, phenethyl resorcinol, acacetin, eriodictyol, phloretin, 2-phenylethyl 2-aminobenzoate, aromadedrin, benzoyl peroxide, heliotropyl phenyl acetate, chalcone, benzyl para-anisate, cinnamyl anthranilate, (Z)-2-penten-1-yl benzoate, 2-naphthyl phenyl ketone, bergamottin, isoimperatorin, para-cresyl benzoate, guaiacyl benzoate, caffeic acid ethyl ester, 4-phenyl propyl pyridine, 2-phenylethyl benzoate, benzyl benzoate, 2-methyl butyl salicylate, phenethyl sorbate, para-anisaldehyde, methyl anthranilate schiff's base, bibenzyl ether, ortho-anisyl benzoate, cinnamyl benzoate, 1-(2-hydroxy-4-methoxyphenyl)-3-(pyridin-2-yl)propan-1-one, ethyl 4-methylphenoxyacetate, benzyl trans-2-methyl-2-butenoate, alpha, alpha-dimethyl anisyl acetone, styryl acetate, amyl cinnamate, ortho-cresyl benzoate, methyl phenyl methylene aminobenzoate, phenethyl pyridine, (Z)-N-coumaroyl-5-hydroxyanthranilic acid, homoeriodictyol, benzyl 2,3-dimethyl-2-butenoate, meta-cresyl benzoate, (Z)-anisylidene acetone, guaiacyl phenyl acetate, 3-phenyl propyl salicylate, geranyl cantabiline, cortex pyridine, ethyl para-cresyl carbonate, ethyl phenoxyacetate, hesperetin, 3′,7-dihydroxy-4′-methoxyflavan, allyl (Z)-cinnamate, bisdemethoxycurcumin, amyl salicylate, phenyl acetaldehyde, methyl anthranilate schiff's base, p-tolyl phenylacetate, 4-formyl phenyl butyrate, 4-methoxybenzyl phenylacetate, para-cresyl 3-oxo-3-phenyl propionate, (E)-2-hexen-1-yl salicylate, furfuryl phenyl acetate, butyl 2-naphthyl ether, (E)-cinnamyl acetate, cinnamyl acetate, ethyl (E)-cinnamate, ethyl cinnamate, 2-para-cresyl oxyethyl acetate, neryl anthranilate, cinnamaldehyde, methyl anthranilate schiff's base, cinnamyl tiglate, (E)-cinnamyl propionate, 1-(4-Methoxyphenyl)-1-penten-3-one, imperatorin, para-cresyl valerate, meta-cresyl phenyl acetate, 3-methyl-3-buten-1-yl benzoate, para-anisyl acetoacetate, isoamyl p-methoxycinnamate, isoeugenyl benzoate, (Z)-3-hexen-1-yl cinnamate, eugenyl benzoate, isoamyl salicylate, phenylmethyl benzeneacetate, curcumin, butyl cinnamate, cinnamyl acetoacetate, para-cresyl phenyl ether, citral, methyl anthranilate schiff's base, cinnamyl valerate, xanthohumol, allyl phenoxyacetate, 2-phenylethyl 3-phenyl-2-propenoate, geranyl benzoate, (E)-3-butylidene phthalide, geranyl cinnamate, neryl cinnamate, cinnamyl cinnamate, amyl anthranilate, (E)-2-hexen-1-yl formate, (E)-3-hexen-1-yl formate, (E)-9-tetradecen-1-yl formate, (E)-cinnamyl formate, (E,Z)-2,6-nonadien-1-yl formate, (Z)-3-hexen-1-yl formate, (Z)-9-tetradecen-1-yl formate, 1,3,4,5,6,7-hexahydro-1,1,5,5-tetramethyl-2H-2,4a-methanonaphthalen-7-yl formate, 1-hexyl allyl formate, 2,6-dimethylhept-5-enyl formate, 2-decalinyl formate, 2-melozol formate, 2-methoxyethyl formate, 2-methyl butyl formate, 2-naphthyl formate, 2-octyl formate, 2-pentyl formate, 2-phenoxyethyl formate, 3-cyclohexyl 3-hexenyl formate, 3-mercapto-3-methyl butyl formate, 3-octyl formate, 3-phenyl propyl formate, 4-tert-amyl cyclohexyl formate, 4-tert-butyl cyclohexyl formate, allyl formate, alpha-amyl cinnamyl formate, alpha-campholenyl formate, aluminum triformate, amber formate, ammonium formate, amyl formate, benzyl formate, bornyl formate, butyl chloroformate, butyl formate, calcium formate, carvyl formate, caryophyllene formate, cedryl formate, chitosan formate, cholesteryl formate, cinnamyl formate, citronellyl formate, cuminyl formate, cyclodecyl formate, cyclododecyl formate, cyclohexyl formate, cyclooctyl formate, cymbopogon martini herb extract formate, decahydro-1,1,7-trimethyl-3a, 7-methano-3ah-cyclopentacyclooct-3-yl formate, decyl formate, dextro-bornyl formate, dihydrocarvyl formate, dihydrocitronellyl formate, dihydromyrcenyl formate, dimethyl benzyl carbinyl formate, dodecyl formate, ethyl 4-methyl-5-thiazolyl formate, ethyl benzoyl formate, ethyl chloroformate, ethyl formate, ethyl orthotrithioformate, ethyl vanillyl formate, eugenyl formate, fenchyl formate, furfuryl formate, geranyl formate, heliotropyl formate, heptyl formate, herbal cyclohexane formate, hexahydromethanoindenyl formate, hexahydrotetramethyl methanonaphthalene-8-methyl formate, hexyl formate, isoamyl formate, isobornyl formate, isobutyl formate, isodecyl formate, isoeugenyl formate, isononyl formate, isopropyl formate, isopulegyl formate, laevo-menthyl formate, linalyl formate, longifolene formate, marine formate, melozol formate, menthadienyl formate, menthyl formate, methyl formate, myristyl formate, myrtenyl formate, neryl formate, nonan-3-yl formate, nonisyl formate, nonyl formate, nopyl formate, octyl formate, oleyl formate, (±)-3-methyl-gamma-decalactone, (±)-chrysanthemolactone, (±)-dihydromint lactone, (±)-isoalantolactone, (±)-pantolactone, (±)-trans-whiskey lactone, (+/−)-mint lactone, (−)-cis-whiskey lactone, (−)-isomint lactone, (−)-mint lactone, (1R)-chrysanthemolactone, (1S)-chrysanthemolactone, (E)-dairy lactone, (R)-(−)-pantolactone, (R)-delta-decalactone, (R)-delta-dodecalactone, (R)-delta-hexalactone, (R)-delta-nonalactone, (R)-delta-octalactone, (R)-delta-undecalactone, (R)-epsilon-decalactone, (R)-gamma-decalactone, (R)-gamma-dodecalactone, (R)-gamma-heptalactone, (R)-gamma-hexalactone, (R)-gamma-nonalactone, (R)-gamma-octalactone, (R)-gamma-undecalactone, (R)-gamma-valerolactone, (R)-massoia lactone, (S)-delta-decalactone, (S)-delta-dodecalactone, (S)-delta-hexalactone, (S)-delta-nonalactone, (S)-delta-octalactone, (S)-delta-undecalactone, (S)-epsilon-decalactone, (S)-gamma-decalactone, (S)-gamma-dodecalactone, (S)-gamma-heptalactone, (S)-gamma-hexalactone, (S)-gamma-nonalactone, (S)-gamma-octalactone, (S)-gamma-undecalactone, (S)-gamma-valerolactone, (S)-massoia lactone, (Z)-dairy lactone, (Z)-gamma-jasmolactone, (Z)-yuzu lactone, (Z,E)-nepetalactone, 1beta-hydroxyalantolactone, 2-acetyl butyrolactone, 2-C-methyl-1,4-erythrono-D-lactone, 2-decen-1,4-lactone, 2-hydroxyalantolactone, 2-isobutyl-gamma-valerolactone, 23-hydroxyphysalolactone, 3-isobutenyl-5-methyl-5-vinyl butyrolactone, 3-methyl butyrolactone, 4-ethyl-gamma-octalactone, 4-hydroxy-3-(methylthio) butanoic acid lactone, 5-hydroxy-7-decenoic acid lactone, 9-hydroxy-gamma-tetradecalactone, alantolactone, alpha-angelica lactone, alpha-decalactone, alpha-methyl-gamma-butyrolactone, animal carbolactone, beta-angelica lactone, beta-methyl-delta-valerolactone, beta-propiolactone, blumealactone A, blumealactone B, blumealactone C, butyrolactonethiol, cassialactone, catalpalactone, cis,cis-nepetalactone, cis-oak lactone, costus lactone, costus valerolactone, creamy lactone, curcumalactone, D-mevalonolactone. dairy lactone, dehydrocostus lactone, dehydrodiferulic dilactone, dehydroindicolactone, delta-2-dodecenolactone, delta-decalactone, delta-dodecalactone, delta-gluconolactone, delta-heptalactone, delta-hexalactone, delta-juniper lactone, delta-nonalactone, delta-octadecalactone, delta-octalactone, or delta-tetradecalactone or a mixture thereof.

9. A method of treating, or delaying the progression of, a cancer or Alzheimer's disease, improving immunity against infectious disease, or reducing inflammation in an animal in need thereof, comprising administering to the animal, an effective amount of a compound selected from the group consisting of alpha-angelica lactone, 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, diacetyl, and methylglyoxal, or a mixture thereof.

10. The method of claim 9, wherein the cancer is a neurological cancer.

11. The method of claim 9, wherein the cancer is neuroblastoma, glioblastoma, prostate cancer or lung cancer.

12. The method of claim 9, wherein the cancer is dependent on the oncogene Nmyc expression.

13. The method of claim 9, wherein the infectious disease is caused by a bacteria, virus or fungus.

14. A method of modulating gene expression in brain neurons, past the blood brain barrier, in an animal in need thereof, comprising administering to the animal, an effective amount of a compound selected from the group consisting of alpha-angelica lactone, 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, diacetyl, and methylglyoxal, or a mixture thereof.

15. A method of improving an immune response in an animal in need thereof, comprising administering to the animal, an effective amount of a compound selected from the group consisting of alpha-angelica lactone, 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, diacetyl, and methylglyoxal, or a mixture thereof.

16. A method of decreasing inflammation in an animal in need thereof, comprising administering to the animal an effective amount of a compound selected from the group consisting of alpha-angelica lactone, 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, diacetyl, and methylglyoxal, or a mixture thereof.

17-18. (canceled)

19. The method of claim 9, wherein the compound is diacetyl.

20-21. (canceled)

22. A pharmaceutical composition, comprising 3,4-hexanedione, methyl pyruvate, ethyl pyruvate, propyl formate, allyl butyrate, 2,3-hexanedione, 2,3-heptanedione, 1-acetoxyacetone, butanol, furfuryl alcohol, 3-heptanone, 2,3-butanediol, 2,3-pentanedione, (E)-tiglaldehyde, allyl acetone, mesityl oxide, 3-methyl-2-buten-1-al, acetoin, methyl propionate, ethyl acetate, allyl propionate, ethyl propionate, ethyl thioacetate, acetone, methylglyoxal, benzyl nicotinate, a flavone, phenethyl resorcinol, acacetin, eriodictyol, phloretin, 2-phenylethyl 2-aminobenzoate, aromadedrin, benzoyl peroxide, heliotropyl phenyl acetate, chalcone, benzyl para-anisate, cinnamyl anthranilate, (Z)-2-penten-1-yl benzoate, 2-naphthyl phenyl ketone, bergamottin, isoimperatorin, para-cresyl benzoate, guaiacyl benzoate, caffeic acid ethyl ester, 4-phenyl propyl pyridine, 2-phenylethyl benzoate, benzyl benzoate, 2-methyl butyl salicylate, phenethyl sorbate, para-anisaldehyde, methyl anthranilate schiff's base, bibenzyl ether, ortho-anisyl benzoate, cinnamyl benzoate, 1-(2-hydroxy-4-methoxyphenyl)-3-(pyridin-2-yl) propan-1-one, ethyl 4-methylphenoxyacetate, benzyl trans-2-methyl-2-butenoate, alpha, alpha-dimethyl anisyl acetone, styryl acetate, amyl cinnamate, ortho-cresyl benzoate, methyl phenyl methylene aminobenzoate, phenethyl pyridine, (Z)-N-coumaroyl-5-hydroxyanthranilic acid, homoeriodictyol, benzyl 2,3-dimethyl-2-butenoate, meta-cresyl benzoate, (Z)-anisylidene acetone, guaiacyl phenyl acetate, 3-phenyl propyl salicylate, geranyl cantabiline, cortex pyridine, ethyl para-cresyl carbonate, ethyl phenoxyacetate, hesperetin, 3′,7-dihydroxy-4′-methoxyflavan, allyl (Z)-cinnamate, bisdemethoxycurcumin, amyl salicylate, phenyl acetaldehyde, methyl anthranilate schiff's base, p-tolyl phenylacetate, 4-formyl phenyl butyrate, 4-methoxybenzyl phenylacetate, para-cresyl 3-oxo-3-phenyl propionate, (E)-2-hexen-1-yl salicylate, furfuryl phenyl acetate, butyl 2-naphthyl ether, (E)-cinnamyl acetate, cinnamyl acetate, ethyl (E)-cinnamate, ethyl cinnamate, 2-para-cresyl oxyethyl acetate, neryl anthranilate, cinnamaldehyde, methyl anthranilate schiff's base, cinnamyl tiglate, (E)-cinnamyl propionate, 1-(4-Methoxyphenyl)-1-penten-3-one, imperatorin, para-cresyl valerate, meta-cresyl phenyl acetate, 3-methyl-3-buten-1-yl benzoate, para-anisyl acetoacetate, isoamyl p-methoxycinnamate, isoeugenyl benzoate, (Z)-3-hexen-1-yl cinnamate, eugenyl benzoate, isoamyl salicylate, phenylmethyl benzeneacetate, curcumin, butyl cinnamate, cinnamyl acetoacetate, para-cresyl phenyl ether, citral, methyl anthranilate schiff's base, cinnamyl valerate, xanthohumol, allyl phenoxyacetate, 2-phenylethyl 3-phenyl-2-propenoate, geranyl benzoate, (E)-3-butylidene phthalide, geranyl cinnamate, neryl cinnamate, cinnamyl cinnamate, amyl anthranilate, (E)-2-hexen-1-yl formate, (E)-3-hexen-1-yl formate, (E)-9-tetradecen-1-yl formate, (E)-cinnamyl formate, (E,Z)-2,6-nonadien-1-yl formate, (Z)-3-hexen-1-yl formate, (Z)-9-tetradecen-1-yl formate, 1,3,4,5,6,7-hexahydro-1,1,5,5-tetramethyl-2H-2,4a-methanonaphthalen-7-yl formate, 1-hexyl allyl formate, 2,6-dimethylhept-5-enyl formate, 2-decalinyl formate, 2-melozol formate, 2-methoxyethyl formate, 2-methyl butyl formate, 2-naphthyl formate, 2-octyl formate, 2-pentyl formate, 2-phenoxyethyl formate, 3-cyclohexyl 3-hexenyl formate, 3-mercapto-3-methyl butyl formate, 3-octyl formate, 3-phenyl propyl formate, 4-tert-amyl cyclohexyl formate, 4-tert-butyl cyclohexyl formate, allyl formate, alpha-amyl cinnamyl formate, alpha-campholenyl formate, aluminum triformate, amber formate, ammonium formate, amyl formate, benzyl formate, bornyl formate, butyl chloroformate, butyl formate, calcium formate, carvyl formate, caryophyllene formate, cedryl formate, chitosan formate, cholesteryl formate, cinnamyl formate, citronellyl formate, cuminyl formate, cyclodecyl formate, cyclododecyl formate, cyclohexyl formate, cyclooctyl formate, cymbopogon martini herb extract formate, decahydro-1,1,7-trimethyl-3a, 7-methano-3ah-cyclopentacyclooct-3-yl formate, decyl formate, dextro-bornyl formate, dihydrocarvyl formate, dihydrocitronellyl formate, dihydromyrcenyl formate, dimethyl benzyl carbinyl formate, dodecyl formate, ethyl 4-methyl-5-thiazolyl formate, ethyl benzoyl formate, ethyl chloroformate, ethyl formate, ethyl orthotrithioformate, ethyl vanillyl formate, eugenyl formate, fenchyl formate, furfuryl formate, geranyl formate, heliotropyl formate, heptyl formate, herbal cyclohexane formate, hexahydromethanoindenyl formate, hexahydrotetramethyl methanonaphthalene-8-methyl formate, hexyl formate, isoamyl formate, isobornyl formate, isobutyl formate, isodecyl formate, isoeugenyl formate, isononyl formate, isopropyl formate, isopulegyl formate, laevo-menthyl formate, linalyl formate, longifolene formate, marine formate, melozol formate, menthadienyl formate, menthyl formate, methyl formate, myristyl formate, myrtenyl formate, neryl formate, nonan-3-yl formate, nonisyl formate, nonyl formate, nopyl formate, octyl formate, oleyl formate, (±)-3-methyl-gamma-decalactone, (±)-chrysanthemolactone, (±)-dihydromint lactone, (±)-isoalantolactone, (±)-pantolactone, (±)-trans-whiskey lactone, (+/−)-mint lactone, (−)-cis-whiskey lactone, (−)-isomint lactone, (−)-mint lactone, (1R)-chrysanthemolactone, (1S)-chrysanthemolactone, (E)-dairy lactone, (R)-(−)-pantolactone, (R)-delta-decalactone, (R)-delta-dodecalactone, (R)-delta-hexalactone, (R)-delta-nonalactone, (R)-delta-octalactone, (R)-delta-undecalactone, (R)-epsilon-decalactone, (R)-gamma-decalactone, (R)-gamma-dodecalactone, (R)-gamma-heptalactone, (R)-gamma-hexalactone, (R)-gamma-nonalactone, (R)-gamma-octalactone, (R)-gamma-undecalactone, (R)-gamma-valerolactone, (R)-massoia lactone, (S)-delta-decalactone, (S)-delta-dodecalactone, (S)-delta-hexalactone, (S)-delta-nonalactone, (S)-delta-octalactone, (S)-delta-undecalactone, (S)-epsilon-decalactone, (S)-gamma-decalactone, (S)-gamma-dodecalactone, (S)-gamma-heptalactone, (S)-gamma-hexalactone, (S)-gamma-nonalactone, (S)-gamma-octalactone, (S)-gamma-undecalactone, (S)-gamma-valerolactone, (S)-massoia lactone, (Z)-dairy lactone, (Z)-gamma-jasmolactone, (Z)-yuzu lactone, (Z,E)-nepetalactone, 1beta-hydroxyalantolactone, 2-acetyl butyrolactone, 2-C-methyl-1,4-erythrono-D-lactone, 2-decen-1,4-lactone, 2-hydroxyalantolactone, 2-isobutyl-gamma-valerolactone, 23-hydroxyphysalolactone, 3-isobutenyl-5-methyl-5-vinyl butyrolactone, 3-methyl butyrolactone, 4-ethyl-gamma-octalactone, 4-hydroxy-3-(methylthio) butanoic acid lactone, 5-hydroxy-7-decenoic acid lactone, 9-hydroxy-gamma-tetradecalactone, alantolactone, alpha-angelica lactone, alpha-decalactone, alpha-methyl-gamma-butyrolactone, animal carbolactone, beta-angelica lactone, beta-methyl-delta-valerolactone, beta-propiolactone, blumealactone A, blumealactone B, blumealactone C, butyrolactonethiol, cassialactone, catalpalactone, cis,cis-nepetalactone, cis-oak lactone, costus lactone, costus valerolactone, creamy lactone, curcumalactone, D-mevalonolactone. dairy lactone, dehydrocostus lactone, dehydrodiferulic dilactone, dehydroindicolactone, delta-2-dodecenolactone, delta-decalactone, delta-dodecalactone, delta-gluconolactone, delta-heptalactone, delta-hexalactone, delta-juniper lactone, delta-nonalactone, delta-octadecalactone, delta-octalactone, or delta-tetradecalactone and a pharmaceutically acceptable excipient.

23-24. (canceled)

25. The method of claim 14, wherein the compound is diacetyl.

26. The method of claim 15, wherein the compound is diacetyl.

27. The method of claim 16, wherein the compound is diacetyl.
